(19) United States
(12) Patent Application Publication
(10) Pub. No.: US 2006/0019264 A1
(43) Pub. Date: Jan. 26, 2006

(54) Method for isolation of independent, parallel chemical micro-reactions using a porous filter

(76) Inventors: Said Attiya, New Haven, CT (US); Vinod B. Makhijani, Guilford, CT (US); Ming Lei, Madison, CT (US); Yi-Ju Chen, New Haven, CT (US); John Simpson, Madison, CT (US); C. Thomas Roth, Fairfield, CT (US); Chun Heen Ho, East Haven, CT (US)

Correspondence Address: MINTZ LEVIN COHN FERRIS GLOVSKY & POPEO, 666 THIRD AVENUE, NEW YORK, NY, 10017, US

(21) Appl. No.: 11/016,942

(22) Filed: Nov. 23, 2004

Related U.S. Application Data
(60) Provisional application No. 60/526,160, filed on Dec. 1, 2003.

(51) Int. Cl. C12Q 1/68; C12M 1/34

(52) U.S. Cl. 435/6; 435/287.2

(57) ABSTRACT

The present invention relates to methods and apparati for conducting densely packed, independent chemical reactions in parallel in a substantially two-dimensional array. Accordingly, this invention also focuses on the use of this array for applications such as DNA sequencing, most preferably pyrosequencing, and DNA amplification.

### RELATED APPLICATIONS

This application claims the benefit of U.S. Application Ser. No. 60/526,160 filed Dec. 1, 2003, which is hereby incorporated by reference herein in its entirety.

### FIELD OF THE INVENTION

The invention describes methods and apparati for conducting densely packed, independent chemical reactions in parallel in a membrane reactor array with mobile solid supports disposed thereon.

### BACKGROUND OF THE INVENTION

High throughput chemical synthesis and analysis are rapidly growing segments of technology for many areas of human endeavor, especially in the fields of material science, combinatorial chemistry, pharmaceuticals (e.g., drug synthesis, testing), and biotechnology (e.g., DNA sequencing, genotyping).

Increasing throughput in any such process requires either that individual steps of the process be performed more quickly, with emphasis placed on accelerating rate-limiting steps, or that larger numbers of independent steps be performed in parallel. One approach for conducting chemical reactions in a high-throughput manner includes performing larger numbers of independent steps in parallel, specifically conducting simultaneous, independent reactions with a multi-reactor system.

A common format for conducting parallel reactions at high throughput comprises two-dimensional (2-D) arrays of individual reactor vessels, such as the 96-well or 384-well microtiter plates widely used in molecular biology, cell biology, and other areas. Individual reagents, solvents, catalysts, and the like are added sequentially and/or in parallel to the appropriate wells in these arrays, and multiple reactions subsequently proceed in parallel. Individual wells may be further isolated from adjacent wells and/or from the environment Individual wells may be further isolated from adjacent wells and/or from the environment by sealing means (e.g., a tight-fitting cover or adherent plastic sheet) or they may remain open. The base of the wells in such microtiter plates may or may not be provided with filters of various pore sizes.

Further increasing the number of microvessels or microreactors incorporated in such 2-D arrays has been a focus of much research. This has been and is being accomplished by miniaturization. For instance, the numbers of wells that can be molded into plastic microtiter plates has steadily increased in recent years—from 96, to 384, and to 1536. Efforts to further increase the density of wells are ongoing (e.g. Matsuda and Chung, 1994; Michael et al., 1998; Taylor and Walt, 1998).

Attempts to make arrays of microwells and microvessels for use as microreactors has also been a focal point for development in the areas of microelectromechanical and micromachined systems, applying and leveraging some of the microfabrication techniques originally developed for the microelectronics industry (see Matsuda and Chung, 1994; Rai-Choudhury, 1997; Madou, 1997; Cherukuri et al., 1999; Kane et al. 1999; Anderson et al., 2000; Dannoux et al., 2000; Deng et al., 2000; Zhu et al., 2000; Ehrfeld et al., 2000).

Yet another widely applied approach for conducting miniaturized and independent reactions in parallel involves spatially localizing or immobilizing at least some of the participants in a chemical reaction on a surface. This creates large 2-D arrays of immobilized reagents. Reagents immobilized in such a manner include chemical reactants, catalysts, other reaction auxiliaries, and adsorbent molecules capable of selectively binding to complementary molecules. For purposes of this patent specification, the selective binding of one molecule to another - whether reversible or irreversible—will be referred to as a reaction process, and molecules capable of binding in such a manner will be referred to as reactants. Immobilization may be arranged to take place on any number of substrates, including planar surfaces and/or high surface area and sometimes porous support media such as beads or gels. Microarray techniques involving immobilization on planar surfaces have been commercialized for the hybridization of oligonucleotides (e.g. by Affymetrix, Inc.) and for target drugs (e.g. by Graffinity, AB).

A major obstacle to creating microscopic, discrete centers for localized reactions is that restricting unique reactants and products to a single, desired reaction center is frequently difficult. There are two aspects to this problem. The first is that “unique” reagents—i.e., reactants and other reaction auxiliaries that are meant to differ from one reaction center to the next—must be dispensed or otherwise deployed to particular reaction centers and not to their nearby neighbors. Such “unique” reagents are to be distinguished from “common” reagents like solvents, which frequently are meant to be brought into substantial contact with all the reaction centers simultaneously and in parallel. The second aspect of this problem has to do with restricting reaction products to the vicinity of the reaction center where they were created—i.e., preventing them from traveling to other reaction centers with attendant loss of reaction fidelity.

To solve the first problem, reaction centers can consists of discrete microwells with the microvessel walls (and cover, if provided) designed to prevent fluid contact with adjacent microwells. However, delivery of reagents to individual microwells can be difficult, particularly if the wells are especially small. For example, a reactor measuring 100 μm×100 μm×100 μm has a volume of only 1 nanoliter. This can be considered a relatively large reactor volume in many types of applications. Even so, reagent addition in this case requires that sub-nanoliter volumes be dispensed with a spatial resolution and precision of at least ±50 μm. Furthermore, addition of reagents to multiple wells must be made to take place in parallel, since sequential addition of reagents to at most a few reactors at a time would be prohibitively slow. Schemes for parallel addition of reagents with such fine precision exist, but they entail some added complexity and cost.

On the other hand, the reaction centers can be brought into contact with a common fluid, e.g., such that microwells all open out onto a common volume of fluid at some point during the reaction or subsequent processing steps. However, this can cause the reaction products (and excess and/or unconverted reactants) originating in one reaction microwell or vessel to travel and contaminate adjacent reaction microwells. Such cross-contamination of reaction centers can occur (i) via bulk convection of solution containing reactants and products from the vicinity of one well to another, (ii) by diffusion (especially over reasonably short distances) of reactant and/or product species, or (iii) by both processes occurring simultaneously.

In certain cases, the individual chemical compounds that are produced at the discrete reaction centers are themselves the desired objective of the process (e.g., as is the case in combinatorial chemistry). For such compounds, any reactant and/or product cross-contamination that may occur will reduce the yield and ultimate chemical purity of this “library” of discrete products. In other cases, the reaction process is conducted with the objective of obtaining information of some type, e.g., information as to the sequence or composition of DNA, RNA, or protein molecules. For these reactions, the integrity, fidelity, and signal-to-noise ratio of that information may be compromised by chemical “cross-talk” between adjacent or even distant microwells.

The issue of contamination of a reaction center or well by chemical products being generated at nearby reaction centers or microwells becomes even more problematic when reaction sites are arrayed on a 2-D surface (or wells are arranged in an essentially two-dimensional microtiter plate) over which fluid flows. In such situations, compounds produced at a surface reaction site or within a well undergo diffusive transport up and away from the surface (or out of the reaction wells), where they are subsequently swept downstream by convective transport of fluid that is passing through a flow channel in fluid communication with the top surface of the array.

### SUMMARY OF THE INVENTION

A novel technique for densely packing microreactors in a substantially 2-D arrangement is described here. This technique provides not only dense, two-dimensional packing of reaction sites, microvessels, and reaction wells, but also provides for efficient delivery of reagents and removal of products by convective flow rather than by diffusion alone. This latter feature permits much more rapid delivery of reagents and other reaction auxiliaries. In addition, it permits faster and more complete removal of reaction products and by-products than has heretofore been possible using methods and apparatus described in the prior art.

One embodiment of the invention is directed to a membrane reactor array comprising a porous supporting membrane layer attached to a planar mesh array. The planar mesh comprises a plurality of pores with reagent-carrying beads of an appropriate size disposed in the pores. As an example, an appropriate size is one whereby the beads are retained in the pores of the mesh. The mesh array is permeable to an aqueous fluid, such as a fluid or reagent used in sequencing but the mesh array is not permeable to the reagent-carrying beads. In a preferred embodiment, the planar mesh array is weaved from individual fibers with a spacing of less than about 100 μm center to center. In another preferred embodiment, the weaving may be made from two sets of parallel fibers that intersect at right angles. In other words, the weaving may be similar to the strings on a tennis racket at a microscopic scale.

Another embodiment of the invention is directed to a membrane reactor array comprising a planar mesh array with a plurality of beads disposed within the pores of the planar mesh array. Each pore has an opening of less than about 40 μm. The pores are sufficiently small such that the planar mesh array is impermeable to the beads. That is, for a planar mesh array with a pore size of 40 μm, each bead should be somewhat greater than 40 μm in diameter. In a preferred embodiment, the beads are 2-3 μm larger than the pore size length. This relationship between bead size and pore size also ensures that only one bead is immobilized to a pore.

Another embodiment of the invention is directed to a method of identifying a base at a target position in one or more sample nucleic acid, preferably DNA, sequences. In one embodiment of the method, a sample DNA sequence and an extension primer, which hybridizes to the sample DNA immediately adjacent to the target position is provided. The DNA sample and extension primer is subjected to a polymerase reaction in the presence of a deoxynucleotide or dideoxynucleotide so that the deoxynucleotide or dideoxynucleotide will only become incorporated and release pyrophosphate (PPᵢ) if it is complementary to the base in the target position. Any release of PPᵢ is detected enzymatically, such as, for example, by detecting a light emission generated by an enzyme in response to the presence of PPᵢ. It should be noted that the light emission may be generated directly or through a chemical pathway involving additional chemical steps or amplification steps. In the method, different deoxynucleotides or dideoxynucleotides are added successively to the sample-primer mixture and subjected to the polymerase reaction to indicate which deoxynucleotide or dideoxynucleotide is incorporated. Further, the sample DNA is immobilized on a bead within a planar membrane reactor array. In a preferred embodiment, the sequencing reaction is a pyrophosphate sequencing reaction. In another preferred embodiment, the sequencing reagents, including the deoxynucleotides or dideoxynucleotides, are contacted to the nucleic acid by a flow of reagent that is normal (orthogonal) to the plane of the planar membrane reactor array. An advantage of any of the methods of this disclosure where the reagent flow is normal to the plane of the membrane reactor array is reduction or elimination of cross contamination. Because the flow is normal to the plane of the beads, each fluid stream will only contact one bead or one species of DNA before it is disposed into a waste container. The chance of cross contamination is reduced significantly.

Another embodiment of the invention is directed to a method of loading a membrane reactor array with nucleic acid. In the method, a planar mesh array that is substantially permeable to a fluid but substantially impermeable to a population of microreactor beads is provided. A fluid comprising a suspension of said population of microreactor beads is introduced onto the surface of the membrane reactor array. The microreactor beads may be linked to a sample nucleic acid or it may be unlinked. Then the beads are settled onto the pores of the membrane reactor array, preferably using a pump or negative pressure or suction. Settling may be performed, for example by allowing the beads to slowly settle out of solution under gravity. Another method of settling may involve centrifugation. In a preferred method, the fluid is drawn through the planar membrane reactor array. Since the beads are bigger than the pores of the planar reactor array, they are trapped (loaded) in the planar reactor array as the fluid is drawn through.

Another embodiment of the invention is directed to a microimaging system for imaging a light emission (e.g., from a pyrophosphate sequencing reaction) from a membrane reactor array. The system comprises two lens groups. The first lens group is the front lens group which is positioned closer to the light source to be detected to collect the light emitted. The second lens group is the rear lens group that is positioned closer to the light detector such as a CCD detection device to image the light onto the detector. In a preferred embodiment, the lens group comprises 50 mm lens with an aperture larger than or equal to 2.8 (e.g., 2.0, 1.8, 1.4, 1.0, etc.). It should be noted that the larger apertures are expressed by a smaller aperture value so that, for example, an aperture of 1 is larger than an aperture of 2.

Another embodiment of the invention is directed to a sequencing cartridge. The cartridge comprises a flow chamber for enclosing an above described membrane reactor array. A membrane supporting structure inside the flow chamber separates the flow chamber into two subchambers. The first subchamber contains the membrane reactor array and also contains an inlet and a first outlet for controlling a fluid flow tangential to the membrane reactor array. The first subchamber also contains a window, covered with a transparent material such as glass or crystal, to allow the optical examination of the membrane reactor array. The second subchamber without the membrane reactor array contains a second outlet allowing fluid to flow normally (orthogonally) from the inlet, through the membrane reactor array, and out through the second outlet. In this manner, both the tangential and normal flow of reagent through the membrane reactor array may be regulated.

Another embodiment of the invention is directed to a method of amplifying a sample nucleic acid on a bead and then loading the bead on a membrane reactor array. In this method, one or more nucleic acid templates to be amplified are individually attached to separate beads to form a population of nucleic acid template-carrying beads. Then the template-carrying beads are suspended in an amplification reaction solution containing reagents necessary to perform nucleic acid amplification. Then, an emulsion is formed to encapsulate the plurality of said template-carrying beads with PCR reaction solution to form a plurality of microreactors (see, e.g., U.S. Application Ser. No. 60/476,504, filed Jun. 6, 2003, which is hereby incorporated by reference in its entirety). In a subsequent step one or more nucleic acid templates in fluidic isolation from each other are amplified to form multiple copies of nucleic acid templates. The amplified nucleic acid templates, still in fluidic isolation, are attached to the beads. Then the beads are loaded into the membrane reactor array.

Another embodiment of the invention is directed to a method of producing a membrane reactor array by providing one or more nucleic acid templates to be amplified wherein a plurality of nucleic acid templates are individually attached to separate beads to form a population of nucleic acid template-carrying beads. Then the beads are loaded onto the membrane reactor array. After loading, the template-carrying beads are contacted to an amplification reaction solution containing reagents necessary to perform nucleic acid amplification. Then, the nucleic acid template is amplified in fluidic isolation from each other to form amplified nucleic acid. Fluidic isolation may be achieved, for example, by removing most fluids from the membrane reactor array and allowing amplification on the fluids that is still in contact with the bead. Furthermore, oil may be added to the membrane reactor array to prevent evaporation during amplification. Any oil may be removed by organic solvents (such as hexane).

### BRIEF DESCRIPTION OF THE DRAWINGS

FIG. 1 represents an integral or physical composite of a microchannel array and a porous membrane barrier forming a membrane reactor array. The flow of fluid through the membrane reactor array carries reaction participants along with the fluid.

FIG. 2 shows a schematic of one version of the convective flow sequencing apparatus described herein.

FIG. 3 shows a membrane reactor array comprising a nylon mesh membrane useful for trapping reagent-carrying beads and one embodiment of the reagent-carrying beads. Shown here are sepharose beads).

FIG. 4 shows the size of the sepharose beads relative to the membrane pores. In panel A, the beads are shown to be swollen in liquid. In contrast, panel B shows how the beads are shrunken when dry.

FIG. 5 represents a membrane holder with a circular optical window; a flow chamber with an inlet port and a first outlet port; a nylon membrane with sepharose beads; a fine pore nylon membrane; a membrane support structure with 1.02 mm holes, 1.35 pitch; and a funnel-shaped collector with a second outlet port.

FIG. 6 shows a membrane support structure with a 50 mm capillary plate with 10 μm holes and a 12 μm pitch for supporting the membrane reactor array.

FIG. 7 shows a schematic of a pyrophosphate-based sequencing method with photon detection.

FIG. 8 shows an automated convective sequencing apparatus.

FIG. 9 shows a sequencing pyrogram indicating the results of sequence analysis.

FIG. 10 shows a sequencing graph. Panel A shows the results for the negative control (no template added), where no sequence was detected. Panel B shows the results for the template, where the correct sequence was detected.

FIG. 11 shows a coupling of amine-primer and amine-biotin to NHS activated sepharose beads.

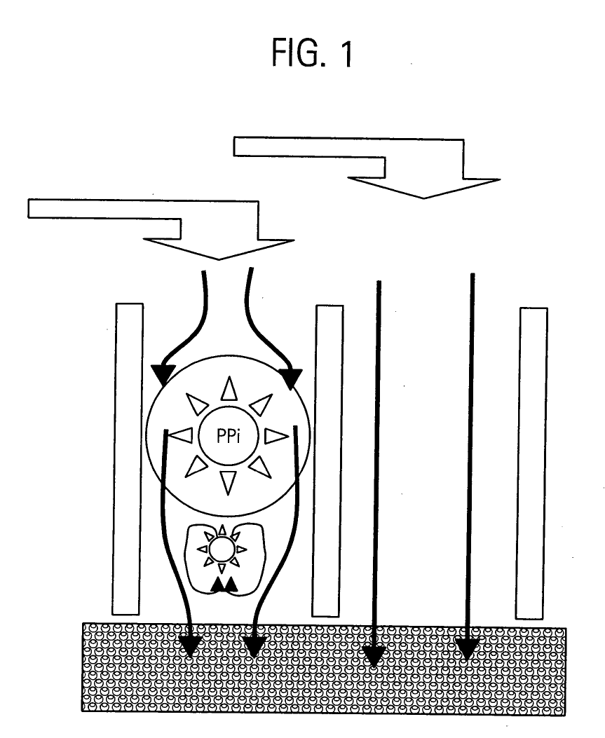

### DETAILED DESCRIPTION OF THE INVENTION

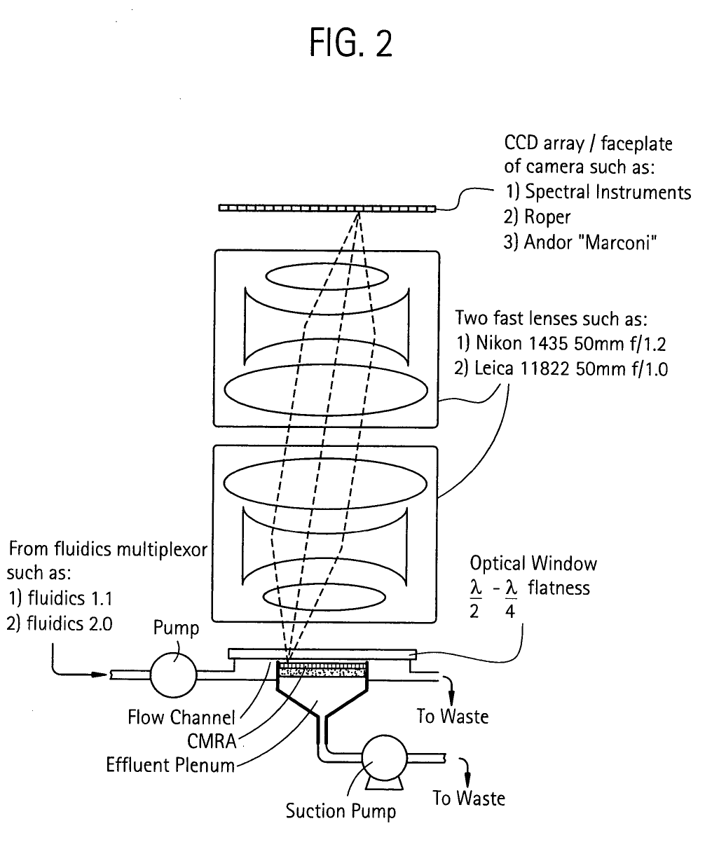

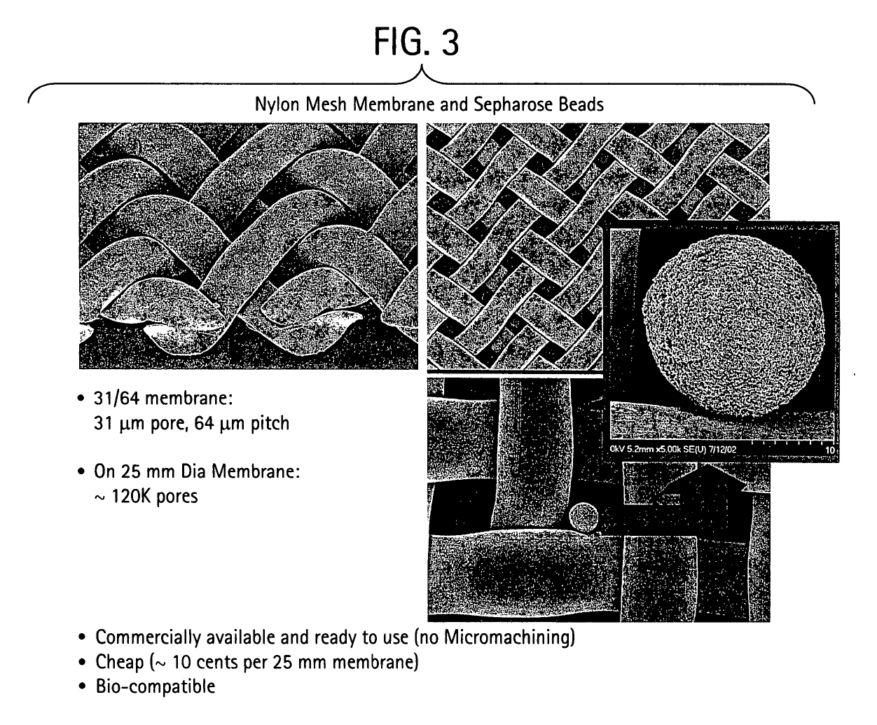

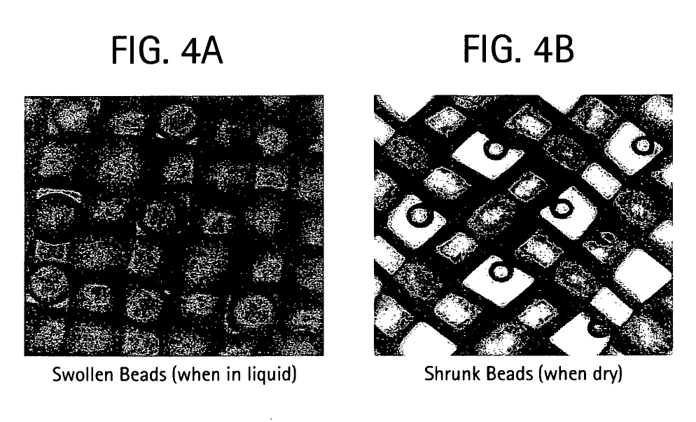

For the purposes of this patent application, the membrane reactor array is a general term that describes both the confined membrane reactor array (CMRA) and the unconfined membrane reactor array (UMRA) as described by U.S. Application Ser. No. 10/191,438 filed Jul. 8, 2002, the entire contents of which are incorporated herein by reference. Methods and apparati are described here for providing a dense array of discrete reaction sites, microreactor vessels, and/or microwells in a substantially two-dimensional configuration (see FIG. 2) and for charging such microreactors with reaction participants by effecting a convective flow of fluid normal to the plane of and through the array of reaction sites or microvessels. The convective flow or delivery of reactants includes both the delivery of sequencing reactants (e.g., dNTPs) towards the reaction site, and convective removal of excess reactants away from the site. Fluid flow sweeps the sequencing reaction products (pyrophosphate (PPᵢ), ATP) through the reaction region in a normal direction thus countering back-diffusion and resultant contamination.

Reaction participants that may be charged, concentrated, and contained within said reaction sites or microreactor vessels by methods of the present invention include high-molecular-weight reactants, catalysts, and other reagents and reaction auxiliaries. In the context of oligonucleotide sequencing and DNA/RNA analysis, such high-molecular-weight reactants include, for example, oligonucleotides, longer DNA/RNA fragments, and constructs thereof. These reactants may be free and unattached (if their molecular weight is sufficient to permit them to be contained by the method of the present invention), or they may be covalently bound to or otherwise associated with, e.g., high-molecular-weight polymers, high-surface-area beads or gels, or other supports known in the art. Examples of reaction catalysts that may be similarly delivered to and localized within said reaction sites or microvessels include enzymes, which may or may not be associated with or bound to solid-phase supports such as porous or non-porous beads. As another example, enzymes such as polymerase may be attached to a support (e.g., beads) as a reagent. In addition, additional polymerase may be delivered to the beads during a reaction to replenish, or supplement the bound polymerase. In this example, a reagent (the polymerase) is both free and unattached and covalently bound or associated with the support. Any reagent of this invention may be both free and bound as described in the above example.

The present invention also includes a means for efficiently supplying relatively lower-molecular-weight reagents and reactants to said discrete reaction sites or microreactor vessels. Also included are means for efficiently removing unconverted reactants and reaction products from said reaction sites or microvessels. More particularly, efficient reagent delivery and product removal are accomplished in the present invention by arranging for at least some convective flow of solution to take place in a direction normal to the plane of the substantially two-dimensional array of reaction sites or microreactor vessels. This flow can lead past or through the discrete sites or microvessels, respectively, where chemical reaction takes place. In this instance, reactants and products will not necessarily be retained or concentrated at the reaction sites or within the reaction microvessels or microwells; indeed, it may be desired that certain reaction products be rapidly swept away from and/or out of said reaction sites or microvessels.

This invention also minimizes the amount of contamination among neighboring reaction sites or “blow-by” which typically occurs in diffusive sequencing. In diffusive sequencing, reaction products from an upstream site have multiple chances to contaminate downstream sites. This contamination is a cumulative effect that may worsen if there are a large number of DNA fragments and multiplets in the upstream reaction sites. In the present invention, the possibility of blow-by has been minimized such that any possible contamination is not cumulative. Specifically, a fluid sheath is formed over each reaction site such that flow downwards relative to the flow laterally is sufficient to prevent blow by or contamination of neighboring reaction sites. Also, each of the beads is washed independently by downward flow of wash solution so that the washing of each reaction site (and any bead disposed therein) is independent of washing of neighboring reaction sites during the washing step.

In addition to including means for providing a controlled convective flux of fluid normal to and across the substantially planar array of reaction sites or microreactors, the present invention also includes permselective, porous filter means capable of discriminating between large (i.e., high-molecular-weight) and small (i.e., low-molecular-weight) reaction participants. This filter means is capable of selectively capturing or retaining certain reaction participants while permitting others to be flushed through and/or out the bottom of the microreactor array. By proper selection of the porous filter and the judicious choice of convective flux rates, considerable control over the location, concentration, and fate of reaction participants can be realized.

Membrane Reactor Arrays

In a preferred embodiment, the apparatus of the present invention consists of an array of microreactor elements comprised of at least two functional elements that may take various physical or structural forms. These include: (i) a planar array layer comprised of an array of microchannels or microwells and on average no more than one reagent-carrying bead or support disposed therein (it is recognized that there may be infrequent times where more than one bead is disposed in a microvessel but this is not preferred), and (ii) a porous high flow resistance membrane comprising, e.g., a porous film or membrane in the form of a sheet or thin layer. These two elements are arranged next to and in close proximity or contact with one another, with the plane of the microchannel/microvessel element parallel to the plane of the porous filter element. In other words, the planar array layer and the porous filter element may be in contact with each other to form one sheet with two layers.

As referred to herein, the side of this composite structure containing the microchannel or microvessel array will be referred to hereinafter as the “top”, while the side defined by the porous filter will be referred to as the “bottom” of the structure. In a preferred embodiment, contact between the planar array and the porous filter may be tight, in which case a fluid cannot travel from the bottom side of one microchannel into another microchannel without entering and exiting the porous filter element. In another embodiment, the contact may also be loose, in which case some fluid may travel from one microchannel to another on the bottom of the planar array layer without passage through the porous filter element. In practice, the flow of fluids in a direction normal to the plane of the membrane reactor array would prevent significant cross contamination between microchannels even if the contacts were loose.

The membrane reactor array will be discussed in more detail following the discussions of each of its component elements.

Planar Array Layer

The planar array layer comprises individual pores (also called microchannel or microvessel) elements. Each pore consists of a single microchannel. The planar array layer comprises a plurality of pores, with the longitudinal axes of said pores being arranged in a substantially parallel manner, and with the downstream ends of said channels being in functional contact with a porous filter element (described below). The aspect ratio of the microchannels (i.e., their height- or length-to-diameter ratio) may be small or large, and their cross-section may take any of a number of shapes (e.g., circular, rectangular, hexagonal, etc.). As discussed further below, it is not at all essential that the microchannel walls be continuous or regular. Highly porous, “spongy” matrices with interconnecting pores communicating between adjacent channels will also serve functionally as microchannel elements, despite the fact that they will not necessarily contain discrete or functionally confined or isolated microchannels per se. In a preferred embodiment, the porous array layer comprises a nylon membrane.

The fundamental requirement is that the effective pore size of the porous array layer be comparable to or smaller than the diameter of the particles that one desires to retain.

In one embodiment, the porous array layer typically contains more than 10,000 pores, preferably more than 50,000 pores, 100,000 pores, or 250,00 pores, and in one preferred embodiment more preferably between about 100,000 and 1,000,000 pores, and in another preferred embodiment, preferably between about 250,000 and 750,000 pores. The porous array layer is typically constructed to have pores with a center-to-center spacing between about 5 to 200 μm, preferably between about 10 to 150 μm, even more preferably between about 25 to 100 μm, and most preferably between about 50 to 78 μm. In five preferred embodiments, the center to center (c-t-c) spacing is about 58 μm, 64 μm, 68 μm, 70 μm, or 100 μm, respectively.

In one embodiment, we contemplate that each reaction chamber in the porous array layer has a pore size length in at least one dimension of between about 5 μm and 200 μm, preferably between about 10 μm and 150 μm, more preferably between about 15 μm and 100 μm, most preferably between about 20 μm and 35 μm. In one embodiment, the reaction chamber can be square and can have the above cited dimensions (or can be rectangular with those dimensions along one linear dimension of the rectangle). For substantially square pores, the average size can include, e.g., about 15 to 100 μm in length, or preferably, about 20 to 35 μm in length. In four preferred embodiments, the reaction chamber is square with pore size lengths of about 25 μm, 28 μm, 30 μm, or 31 μm, respectively.

In four preferred embodiments, the porous array layer is selected from a nylon membrane with: (1) a center to center (c-t-c) spacing of about 64 μm and a 31 μm pore size length; (2) a center to center (c-t-c) spacing of about 58 μm and a 25 μm pore size length; (3) a center to center (c-t-c) spacing of about 70 μm and a 30 μm pore size length; and (4) a center to center (c-t-c) spacing of about 68 μm and a 28 μm pore size length.

It is advantageous to deposit or settle a particular reactant molecule (e.g., an oligonucleotide or construct thereof) at discrete sites on the surface of a membrane reactor array (e.g., for pyrosequencing). This may be accomplished by immobilizing said reactants on particulate or colloidal supports (e.g., beads, particles), suspending the supports in a fluid, and then depositing or settling these onto the membrane reactor array surface by drawing the fluid through the membrane reactor array. One method for depositing a bead is to place a fluid suspension of beads on a membrane reactor array and allowing gravity to deposit the beads into the individual pores—a process that can be accelerated by vibration or centrifugation.

In a preferred embodiment, the planar array is substantially permeable to aqueous solutions but is substantially impermeable to the beads. This is possible, for example, if the pores are smaller than the beads so that beads cannot flow through. In another preferred embodiment, the beads reduce the size of the pores but do not eliminate the opening in the pores. In one embodiment, the beads are spherical while the pores are square (see Examples). The deposition of a round bead in a square pore would still allow a flow of fluid through the pore. In another embodiment, the beads and the pores have irregular shapes that deviate, slightly or grossly, from a perfect sphere and a perfect circle. The deposition of an imperfect spherical bead onto an imperfect circular pore would not completely block the pore. For example, in a preferred embodiment, the planar array layer is a woven mesh of polymer (e.g., nylon or nitrocellulose) fibers. The fibers are woven at right angles to each other to form square pores.

Contemplated for use with the invention are commercially available materials. Preferred materials for the planar array layer of invention include precision woven open mesh fabrics, especially monofilament open mesh fabrics, such as those available from Sefar, Inc. (Ruschlikon, Switzerland). Non-limiting examples of such fabrics include Sefar Nitex (PA 6.6), e.g., Cat. # 03-25/14, 03-28/17, 03-30/18, 03-30/20, and 03-35/16. Other preferred materials for the planar array layer include woven nylon net filters such as those available from Millipore (Bedford, Mass.), including, but not limited to, Cat. # NY41 025 00, NY41 047 00, NY41 090 00, and NY41 000 10.

Porous High Flow Resistance Membrane

The porous high flow resistance filter element provides significant flow restriction in the membrane reactor array. This flow restriction is useful in achieving uniform flow of reagents through the membrane reactor array.

Membrane reactor arrays without a porous high flow resistance membrane element may suffer from a non-uniform flow of reagent. In the absence of a porous high flow resistance membrane, a pore with a bead would permit a flow that is reduced when compared to a pore without a bead. It follows that a mixture of open pores and bead loaded pores in a membrane reactor array would have uneven flow. In a biochemical reaction, an uneven flow may cause some pores to receive reagents in a non-uniform fashion. Non-uniform delivery of reagents may lead, at least, to a delay in performing reactions because a longer flow is necessary to deliver reagents to all the pores. More significantly, non-uniform delivery may cause errors in interpreting results. For example, some pores may receive more reagents than others and the excess or lack of reagents may change the results of a biochemical reaction. Spatially uneven flow through the porous array layer may also result in the lateral diffusion of reaction products from a reactive pore (i.e., one containing a bead) to a neighboring empty pore, which can lead to cross-contamination, or bleed.

The problem with uneven (non-uniform) flow can be reduced to an insignificant level by the use of a porous high flow resistance membrane element. The porous high flow resistance membrane may be configured in any number of ways to provide satisfactory flow resistance in conjunction with the planar array layer. The porous high flow resistance membrane may comprise pores that are less than one tenth ( 1/10) or less than one hundredth ( 1/100) the size of the pores in the planar array layer. The porous high flow resistance membrane, because of its small pores, will have a flow restriction that is about 10-fold or more, preferable about 100-fold or more, than that of the planar array layer. Because the porous high flow resistance membrane provides most of the flow restriction in a membrane reactor array, the pores of the planar array layer, regardless of whether it contains a bead, would provide only a small portion of the flow restriction.

In one embodiment, we contemplate that the porous high flow resistance membrane has an average pore size of between about 0.01 μm and 10 μm, preferably between about 0.01 μm and 5 μm, more preferably between about 0.01 μm and 0.5 μm and even more preferably between about 0.1 μm and 1 μm, or less than 0.1 μm. This is particularly the case when a symmetric membrane is used. In one embodiment, the pore size is about 0.2 μm and in another embodiment, the pore size is about 0.02 μm. Preferably, the high flow resistance membrane has a pore diameter that is less than 10%, or less than 1%, of the pore diameter of the planar array layer.

If the membrane porosity is asymmetric (i.e., an anisotropic membrane) then different pores sizes may also be used.

In another embodiment, membranes with altered surface chemistries may be used. For example, the porous high flow resistance membrane may restrict flow of aqueous materials by being composed of a hydrophobic material. In a preferred embodiment of a hydrophobic porous high flow resistance membrane, we contemplate using a 10-40 μm PTFE membrane. In another preferred embodiment, the membrane has a pore size of less than or about 20 μm. Additionally, membranes with altered surface chemistries may be used (e.g., hydrophobic membranes).

Commercially available materials are contemplated for use with the invention. Preferred materials for the high-flow resistance membrane of the invention include nylon membrane filters such as those available from Millipore, including, but not limited to, Cat. # GNWP 025 00 and GNWP 047 00. Other preferred materials for the high-flow resistance include membrane ceramic filters such as those available from Refractron Technologies Corp. (Newark, N.Y.), including, but not limited to, alumina or silicon carbide filter plates with 15-30 μm pores and 40-50% porosity (volume %).

Optional Porous Structural Support Layer

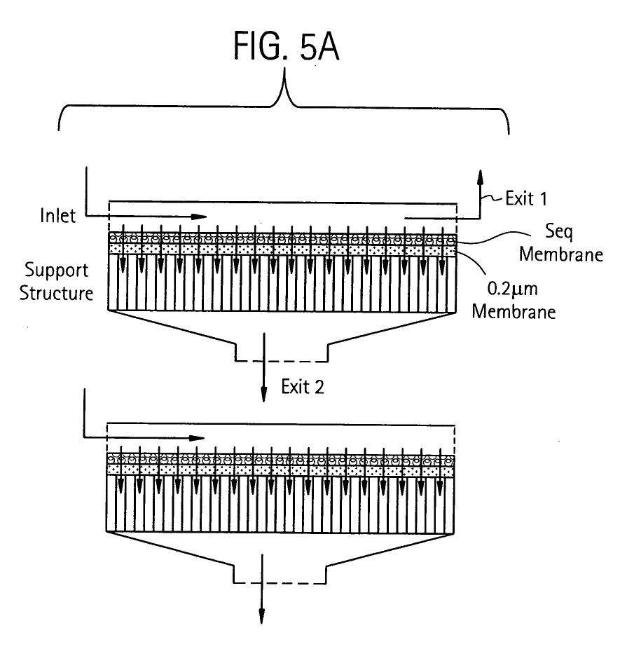

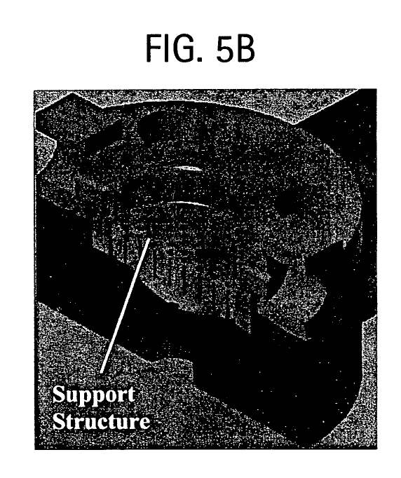

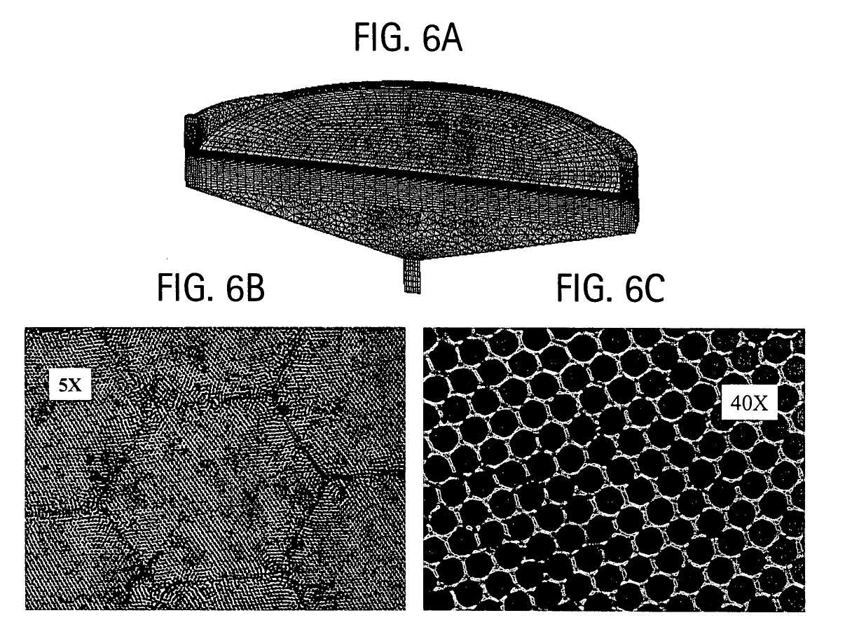

In addition to a planar layer and a porous high flow resistance membrane, the membrane reactor array can also employ a porous structural support layer; that is, more porous than the other two layers and that is, in one embodiment, placed against and/or attached to the high flow resistance membrane. This porous support layer can be used to provide mechanical support to membrane reactor array. See, e.g., FIG. 5 where an example of this is shown.

The porous support layer may be made from any material such as metals, glass, polymers, silicon, ceramics with holes formed during manufacture (e.g., sintering, drilled by laser, cracking, etching, and the like). It is noted that while a nonreactive material is generally preferred for the porous support layer, a nonreactive material is not necessary as long as the flow of reagents from the planar array layer to the porous support layer is sufficiently fast to prevent back diffusion of any molecules from the porous support layer to the planar array layer. In one preferred embodiment, we contemplate that the porous support layer comprises a metal mesh.

In various embodiments, commercially available materials can be used for the support layer. Preferred materials include stainless steel microfiltration meshes, such as Spectra/Mesh® from Spectrum Laboratories (Rancho Dominguez, Calif.), including, but not limited to, Cat. # 145827, 145936, 145826, and 145935.

Membrane Reactor Array Configuration

It should be appreciated that the membrane reactor arrayed can be constructed in a number of different configurations. For example, the porous high flow resistance membrane may be positioned on the top or the bottom of the planar array layer. In addition, two porous high flow resistance membranes may be utilized, with the planar array layer between them. In addition, the porous structural support layer may be positioned in several different configurations, e.g., on the top, bottom or in the middle of the membrane reactor array. As will be appreciated, the porous structural support layer is optional and may not be required when the membrane reactor array is configured with sufficient inherent support (e.g., when the membrane reactor array is provided with additional support by being affixed to a circumferential support, much like a drum head).

In many cases it will be appropriate to consider the entire array assembly (i.e., the combination of microchannel/microvessel element plus porous filter element) as a single substantially two-dimensional structure comprised of either an integral or a physical composite, as described further below. The membrane reactor arrays of the present invention will be seen to possess some of the general structural features and functional attributes of commercially available microtiter filter plates of the sort commonly used in biology laboratories, wherein porous filter disks are molded or otherwise incorporated into the bottoms of plastic wells in 96-well plates. However, the membrane reactor array is differentiated from these by the unparalleled high density of discrete reaction sites that it provides, by its unique construction, and by the novel and uniquely powerful way in which it can be operated to perform high-throughput chemistries—for example, of the sorts applied to the amplification and/or analysis of DNA.

The composite microreactor/filter structure—i.e., the membrane reactor array of the present invention—can take several physical forms; as alluded to above, two such forms are represented by physical composites and integral composites, respectively. The two functional elements of the structure include the planar array layer and the porous high flow resistance membrane. These elements may be provided as separate parts or components that are merely laid side-by-side, pressed together, or otherwise attached in the manner of a sandwich or laminate. This structural embodiment will be referred to hereinafter as a “physical composite”. Additional porous supports (e.g., fine wire mesh or very coarse filters) and/or spacing layers may also be provided where warranted to provide mechanical support. Plastic mesh, wire screening, molded or machined spacers, or similar structures may be provided atop the membrane reactor array to help provide spatial separation between tangential flow of fluid across the top of the membrane reactor array and the upper surface of the membrane reactor array. Similar structures may be provided beneath the membrane reactor array to provide a pathway for egress of fluid that has permeated across the membrane reactor array.

In contrast to the operation of many prior-art microreactor arrays, wherein diffusion of reactants into and products back out of an array of microvessels occurs solely by diffusion, the operation of the membrane reactor arrays of the present invention employs a modest convective flux through the membrane reactor array. In particular, a small pressure difference is applied from the top to the bottom surface of the membrane reactor array sufficient to establish a controlled convective flux of fluid through the structure in a direction normal to the substantially planar surface of the structure. Fluid is thus made to flow first through the microchannel element and then subsequently across the porous filter element. This convective flow enables the rapid delivery to the site of reaction of low-molecular-weight reactants and the efficient and complete removal of low-molecular-weight reaction products from the site of their production. Particularly important is the fact that the convective flow serves to impede or substantially prevent the back-diffusion of reaction products out of the upstream ends of the microchannels, where otherwise they would be capable of contaminating adjacent or even distant microreactor vessels.

In certain reaction systems of interest (e.g., DNA analysis by pyrosequencing, as discussed in more detail below), it may be necessary to avoid covalently immobilizing certain macromolecular reagents altogether. The DNA polymerase used in pyrosequencing is a case in point. It is believed that DNA polymerase should retain at least a certain degree of mobility if it is to function optimally. As a consequence, this particular enzyme must normally be treated as a consumable reagent in pyrosequencing, since it is not desirable to covalently immobilize it and reuse it in subsequent pyrosequencing steps. The polymerase may be in a native form or “tagged” with a moiety that adheres to the beads, such as biotin.

For the polymerase, we contemplate: a) placing polymerase in contact with template loaded beads; b) flowing polymerase over the array; and c) both (i.e., employing (a) and (b) together). However, the present invention provides means for localizing this macromolecular reagent within the microchannels or microvessels of a membrane reactor array without having to covalently immobilize it.

Applications of the Membrane Reactor Array

Many different types of reactions can be performed in a membrane reactor array. In one embodiment, each cavity or reaction chamber of the array contains reagents for analyzing a nucleic acid or protein. Not all reaction chambers are required to include a nucleic acid or protein target. Typically those reaction chambers that contain a nucleic acid contain only a single species of nucleic acid (i.e., a single sequence that is of interest). There may be a single copy of this species of nucleic acid in any particular reaction chamber, or they may be multiple copies. It is generally preferred that a reaction chamber contain at least 1,000,000 copies of the species of nucleic acid sequence of interest, preferably at least between 2,000,000 and 20,000,000 copies, and most preferably between about 5,000,000 to 15,000,000 copies of the species of nucleic acid sequence of interest. In one embodiment the nucleic acid species is amplified to provide the desired number of copies using polymerase chain reaction (“PCR”) (preferred), rolling circle amplification (“RCA”), ligase chain reaction, other isothermal amplification, or other conventional means of nucleic acid amplification. In one embodiment, the nucleic acid is single stranded. In other embodiments the single stranded DNA is a concatamer with each copy covalently linked end to end.

The nucleic acid may be immobilized in the reaction chamber, either by attachment to the chamber itself or preferably by attachment to a mobile solid support (e.g., a bead) that is delivered to the chamber. A bioactive agent could be delivered to the array, by dispersing over the array a plurality of mobile solid supports, each mobile solid support having at least one reagent immobilized thereon, wherein the reagent is suitable for use in a nucleic acid sequencing reaction.

The array can also include a population of mobile solid supports disposed in the reaction chambers, each mobile solid support having one or more bioactive agents (such as a nucleic acid or a sequencing enzyme) attached thereto. The diameter of each mobile solid support can vary, we prefer the diameter of the mobile solid support to be such that only one bead is trapped within a single pore in the sequencing membrane. Not every reaction chamber in the sequencing membrane need contain a mobile solid support. There are numerous contemplated embodiments; in one embodiment, at least 5% to 20% of the reaction chambers can have a mobile solid support having at least one reagent immobilized thereon; a second embodiment has about 20% to 60% of the reaction chambers can have a mobile solid support having at least one reagent immobilized thereon; and a third embodiment has about 50% to 100% of the reaction chambers with a mobile solid support having at least one reagent immobilized thereon. Preferably, the percentage of pores loaded with beads is about 5%, 10%, or 25%.

The mobile solid support typically has at least one reagent immobilized thereon. For the embodiments relating to pyrosequencing reactions or more generally to ATP detection, the reagent may be a polypeptide with sulfurylase or luciferase activity, or both. Alternatively, enzymes such as hypoxanthine phosphoribosyltransferase, xanthine oxidase, uricase or peroxidase could be utilized (e.g., Jansson and Jansson (2002), incorporated herein by reference). The mobile solid supports can be used in methods for dispersing over the array a plurality of mobile solid supports having one or more nucleic sequences or proteins or enzymes immobilized thereon.

In another aspect, the invention involves an apparatus for simultaneously monitoring the array of reaction chambers for light generation, indicating that a reaction is taking place at a particular site. In this embodiment, the reaction chambers are sensors, adapted to contain analytes and an enzymatic or fluorescent means for generating light in the reaction chambers. In this embodiment of the invention, the sensor is suitable for use in a biochemical or cell-based assay. The apparatus also includes an optically sensitive device arranged so that in use the light from a particular reaction chamber would impinge upon a particular predetermined region of the optically sensitive device, as well as means for determining the light level impinging upon each of the predetermined regions and means to record the variation of the light level with time for each of the reaction chamber.

In one specific embodiment, the instrument includes a light detection means having a light capture means and a second fiber optic bundle for transmitting light to the light detecting means. We contemplate one light capture means to be a CCD camera. The second fiber optic bundle is typically in optical contact with the array, such that light generated in an individual reaction chamber is captured by a separate fiber or groups of separate fibers of the second fiber optic bundle for transmission to the light capture means.

The membrane reactor array can be utilized to achieve highly parallel sequencing without electrophorectic separation of DNA fragments and associated sample preparation.

The membrane reactor array can also be used for other uses, e.g., combinatorial chemistry. For detection purposes, an array of photodetectors is utilized for monitoring light producing reactions within the membrane reactor array. In a preferred embodiment, the array of photodetectors is a CCD camera. Another method of detection of discrete reactions within the membrane reactor array is to monitor changes in light absorption as an indicator of a chemical reaction in a membrane reactor array using an array of photodetectors.

Sequencing of DNA via Pyrophosphate Detection

The methods and apparatuses described are generally useful for any application in which the identification of any particular nucleic acid sequence is desired. For example, the methods allow for identification of single nucleotide polymorphisms (SNPs), haplotypes involving multiple SNPs or other polymorphisms on a single chromosome, and transcript profiling. Other uses include sequencing of artificial DNA constructs to confirm or elicit their primary sequence, or to identify specific mutant clones from random mutagenesis screens, as well as to obtain the sequence of cDNA from single cells, whole tissues or organisms from any developmental stage or environmental circumstance in order to determine the gene expression profile from that specimen. In addition, the methods allow for the sequencing of PCR products and/or cloned DNA fragments of any size isolated from any source. The DNA may be genomic DNA or cDNA and may be derived from viral, bacterial, fungal, mammalian, or human (preferably) sources.

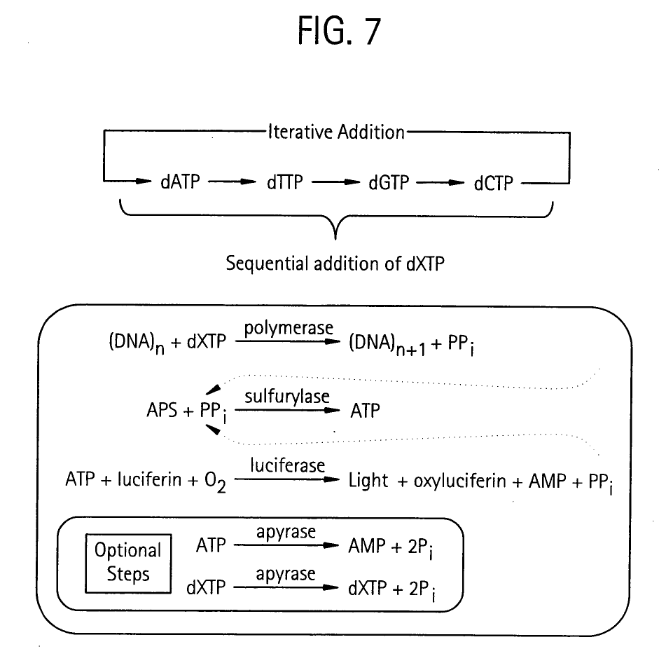

Sequencing of DNA by pyrophosphate detection (“pyrophosphate sequencing”) is described in various patents (Hyman, 1990, U.S. Pat. No. 4,971,903; Nyren et al., U.S. Pat. Nos. 6,210,891 and 6,258,568 and PCT Patent application WO98/13523; Hagerlid et al., 1999, WO99/66313; Rothberg, U.S. Pat. No. 6,274,320, WO01/20039) and publications (Hyman, 1988; Nyrén et al., 1993; Ronaghi et al., 1998, Jensen, 2002; Schuller, 2002). The contents of the foregoing patents, patent applications and publications cited here are incorporated herein by reference in their entireties. Pyrophosphate sequencing is a technique in which a complementary sequence is polymerized using an unknown sequence (the sequence to be determined) as the template. This is, thus, a type of sequencing technique known as “sequencing by synthesis”. Each time a new nucleotide is polymerized onto the growing complementary strand, a pyrophosphate (PPᵢ) molecule is released. This release of pyrophosphate is then detected. Iterative addition of the four nucleotides (dATP, dCTP, dGTP, dTTP) or of analogs thereof (e.g., α-thio-dATP), accompanied by monitoring of the time and extent of pyrophosphate release, permits identification of the nucleotide that is incorporated into the growing complementary strand. A schematic of pyrophosphate based sequencing is shown in FIG. 7.

Pyrophosphate can be detected via a coupled reaction in which pyrophosphate is used to generate ATP from adenosine 5′-phosphosulfate (APS) through the action of the enzyme ATP sulfurylase. The ATP is then detected photometrically via light released by the enzyme luciferase, for which ATP is a substrate. (It may be noted that dATP is added as one of the four nucleotides for sequencing by synthesis and that luciferase can use dATP as a substrate. To prevent light emission on addition of dATP for sequencing, a dATP analog such as α-thio-dATP is substituted for DATP as the nucleotide for sequencing. The α-thio-DATP molecule is incorporated into the growing DNA strand, but it is not a substrate for luciferase.)

Pyrophosphate sequencing can be performed in a membrane reactor array in several different ways. One such protocol follows:
(1) capture DNA whose sequence is to be determined (preferably, many copies of a single sequence) onto beads and load beads onto the membrane reactor array;
(2) for negative control a separate set of beads are prepared as in step (1) with the exception that no DNA is captured. These beads are used along with the DNA loaded beads and serves as a negative control. The negative control is useful, at least, for determining background signal levels; unless specified otherwise, all subsequent steps are performed in parallel using DNA loaded beads and negative control beads;
(3) load: (a) luciferase and sulfurylase onto beads and dispose the loaded beads onto the membrane reactor array (loading beads using any suitable methods of attaching proteins to surfaces known in the art); and (b) DNA polymerase (e.g. Klenow fragment) onto the membrane reactor array (can be done concurrently with step (a) above); and
(4) flow a mixture of dXTP, APS (a substrate for sulfurylase), and luciferin (a substrate for luciferase) through the membrane reactor array, cycling through the four nucleotides (dCTP, dGTP, dTTP, a-thio-dATP, or any suitable dATP analog) one at a time. It will be noted that these are all low-molecular weight molecules, so they will pass through the membrane reactor array without said molecules undergoing appreciable concentration polarization.

The upstream-to-downstream flow of fluid into and through the membrane reactor array thus causes:
(a) addition of the appropriate dXTP (dATP, dTTP, dGTP or dCTP) by the polymerase and attendant production of PPᵢ in the region of the DNA being sequenced (with APS and luciferin flowing through);
(b) production of ATP from APS and PPᵢ when the latter are brought into contact with the sulfurylase enzyme (with luciferin flowing through); and
(c) production of light from ATP and luciferin in the vicinity of the luciferase enzyme.

Light production is then monitored by a photodetector—discussed in more detail below.

For example, a CCD camera, optically coupled by a lens or other means to the membrane reactor array, is capable of monitoring light production simultaneously from many microchannels or discrete reaction sites. CCD cameras are available with millions of pixels, or photodetectors, arranged in a 2-D array. Light originating from one reaction chamber or discrete reaction site in or on a membrane reactor array can be made to strike one or a few pixels on the CCD. Thus, if each reaction chamber or reaction site is arranged to contain and conduct an independent sequencing reaction, each reaction can be monitored by one (or at most a few) CCD elements or photodetectors. By using a CCD camera or other imaging means comprising millions of pixels, the progress of millions of independent sequencing reactions is simultaneously monitored.

Further, each reaction chamber or reaction site can be made to hold the amplification products from only a single strand of DNA, and if different reaction chambers (or bead disposed therein) hold the amplification products of different strands of DNA, then the simultaneous sequencing of millions of different strands of DNA is possible. The distribution of DNA to be sequenced can be accomplished in many ways, two of which follow:
(a) The amplification products of a single oligonucleotide strand are attached to a bead, and beads from many independent amplification reactions are combined and placed onto a membrane reactor array; or
(b) Many different strands of DNA are added in dilute concentration and applied to the membrane reactor array (each strand of DNA separately attached to a different bead) such that many reaction chambers or discrete reaction sites contain only a single strand of DNA. The DNA is then amplified within or upon the membrane reactor array through one series of reactions, and then it is directly sequenced via addition of the reagents described above. One such technique (polymerase chain reaction or PCR) for amplification of DNA within the pores of a membrane reactor array is described below (see Example 2).

Delivery of the DNA to be sequenced and the enzymes and substrates necessary for pyrophosphate-based sequencing can be accomplished in a number of ways.

In a preferred embodiment, one or more reagents are delivered to the membrane reactor array immobilized or attached to a population of mobile solid supports, e.g., a bead or microsphere. The bead or microsphere need not be spherical; irregular shaped beads may be used. The beads are typically constructed from numerous substances, e.g., plastic, glass or ceramic, and cross-linked agarose gel. The bead of the invention may comprise various chemistries, such as, for example, methylstyrene, polystyrene, acrylic polymer, latex, paramagnetic, thoria sol, carbon graphite and titanium dioxide. The construction or chemistry of the bead can be chosen to facilitate the attachment of the desired reagent. In a preferred embodiment, the beads are magnetic or paramagnetic.

Bead sizes depend on the pore size length and width of the reaction chamber. In a preferred embodiment, the diameter of each mobile solid support is chosen so that the mobile solid support cannot pass through the pores (in the membrane reactor array). It should be noted that in other embodiments, the beads may be smaller than the pores—the porous high flow resistance membrane layer can stop the bead from flowing through the membrane reactor array. In a preferred embodiment beads are sized so that only one bead can fit within a single reaction chamber and where the spatial separation between two adjacent reaction chambers has a linear dimension of between about 5 μm and 200 μm, preferably between 10 μm and 150 μm, more preferably between about 25 μm and 100 μm, such as between about 50 μm and 75 μm, and most preferably between about 20 μm and 35 μm. In a specific embodiment, the bead diameter may be 31 μm and the pore diameter may be 33 μm. Even though the 31 μm bead may flow through the 33 μm pore, the porous high flow resistance membrane layer prevents the bead from flowing through.

In some embodiments, one reagent immobilized to the mobile solid support can be a polypeptide with sulfurylase activity, a polypeptide with luciferase activity, or both on the same or different beads, or a chimeric polypeptide having both sulfurylase and luciferase activity. In one embodiment, it can be an ATP sulfurylase and luciferase fusion protein (see, e.g., U.S. patent application Ser. No. 10/122,706, filed Apr. 11, 2002, and U.S. patent application Ser. No 10/154,515, filed May 23, 2002; which are incorporated herein by reference in their entirety). Other sulfurylase and/or luciferase that may be used include those described in U.S. Pat. Nos. 5,583,024; 5,674,713, 5,700,673, and in international application WO 00/24878; all incorporated herein in their entirety. Ultra-Glow luciferase (available from Promega) is also suitable. In a preferred embodiment, both luciferase and sulfurylase are immobilized on the same bead. Since the product of the sulfurylase reaction is consumed by luciferase, proximity between these two enzymes may be achieved by covalently linking the two enzymes in the form of a fusion protein. Alternatively, a fusion protein combining functional polymerase, sulfurylase and luciferase activity may be used.

In other embodiments, the reagent immobilized to the mobile solid support can be the nucleic acid whose sequence is to be determined or analyzed. A DNA or RNA polymerase can be incubated with beads that have nucleic acids attached thereto.

Generally, a membrane reactor array device having normal cross-flow exhibits high levels of wash efficiency. In some cases, proper reactor array membranes and high flow resistance membranes are not available, and the chamber cannot be washed efficiently within a reasonably short period of time. This can have a significant impact on the accuracy of pyrosequencing. In such situations, apyrase may be applied to degrade the leftover nucleotides after each nucleotide delivery. The use of apyrase is typically at concentrations of 1 U/l to 100 U/l, preferably 4 U/l to 40 U/l, most preferably 8 U/l to 20 U/l. In some cases, high fidelity but low processivity polymerase (e.g., Klenow) may have to be used, and in these cases, having polymerase present in the flow may be desirable. Preferably, the flow rate of the membrane reactor array device is about 0.15 ml/minute/cm² to 4 ml/minute/cm², or about 0.1 ml/minute/cm² to 5 ml/minute/cm².

Bead Attachment Chemistry

In some embodiment, the bioactive agents (e.g., nucleic acids) are synthesized, and then covalently attached to the beads. As is appreciated by someone skilled in the art, this will be done depending on the composition of the bioactive agents and the beads. The functionalization of solid support surfaces such as certain polymers with chemically reactive groups such as thiols, amines, carboxyls, etc., is generally known in the art. Accordingly, “blank” beads may be used that have surface chemistries that facilitate the attachment of the desired functionality by the user. Additional examples of these surface chemistries for blank beads include, but are not limited to, amino groups including aliphatic and aromatic amines, carboxylic acids, aldehydes, amides, chloromethyl groups, hydrazide, hydroxyl groups, sulfonates and sulfates.

These functional groups can be used to add any number of different candidate agents to the beads, generally using known chemistries. For example, candidate agents containing carbohydrates may be attached to an amino-functionalized support; the aldehyde of the carbohydrate is made using standard techniques, and then the aldehyde is reacted with an amino group on the surface. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated here by reference) which can be used to attach cysteine containing proteinaceous agents to the support. Alternatively, an amino group on the candidate agent may be used for attachment to an amino group on the surface. For example, a large number of stable bifunctional groups are well known in the art, including homobifunctional and heterobifunctional linkers (see Pierce Catalog and Handbook, pages 155-200).

In an additional embodiment, carboxyl groups (either from the surface or from the candidate agent) may be derivatized using well known linkers (see Pierce catalog). For example, carbodiimides may be used to activate carboxyl groups for attack by good nucleophiles such as amines (see Torchilin et al., Critical Rev. Therapeutic Drug Carrier Systems, 7(4):275-308 (1991)). Proteinaceous candidate agents may also be attached using other techniques known in the art, for example for the attachment of antibodies to polymers; see Slinkin et al., Bioconj. Chem. 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994). It should be understood that the candidate agents may be attached in a variety of ways, including those listed above. Preferably, the manner of attachment does not significantly alter the functionality of the candidate agent; that is, the candidate agent should be attached in such a flexible manner as to allow its interaction with a target.

Specific techniques for immobilizing enzymes on beads are known in the prior art. In one case, NH₂ surface chemistry beads are used. Surface activation is achieved with a 2.5% glutaraldehyde in phosphate buffered saline (10 mM) providing a pH of 6.9 (138 mM NaCl, 2.7 mM KCl). This mixture is stirred on a stir bed for approximately 2 hours at room temperature. The beads are then rinsed with ultrapure water plus 0.01% Tween 20 (surfactant), 0.02%, and rinsed again with a pH 7.7 PBS plus 0.01% Tween 20. Finally, the enzyme is added to the solution, preferably after being prefiltered using a 0.45 μm Amicon™ micropure filter.

One method of preparing beads is as follows:

1. Esterification of carboxyl derivative of sepharose beads is achieved with N-hydroxysuccinimide (NHS) and this leads to the formation of activated esters that react rapidly with primer containing amino-groups to give stable amide bonds.

2. Beads to be used for this purpose are supplied (Amersham) in 100% isopropanol to preserve the activity prior to coupling.

3. 25 μl of 1 mM amine-labeled HEG primer are dissolved in coupling buffer (200 mM NaHCO3, 0.5 M NaCl, pH 8.3).

4. Beads were activated by adding 1 ml of ice cold 1 mM HCl.

5. Beads were washed two times with ice cold coupling buffer.

6. Amine labeled primers and amine labeled biotin, in a ratio of 1:9 respectively) is added to the beads and incubated for 15 to 30 minutes at room temperature with rotation (to allow coupling to happen). Amine-labeled biotin is added so that after coupling the emulsion PCR, the streptavidin is added to be coupled to the biotin. Then the biotinylated sulfurylase and luciferase (454 Life Sciences) will be coupled to the streptavidin.

7. Then the beads were washed one time with coupling buffer.

8. The beads were washed two times with Acetate buffer (0.1 M sodium acetate, 0.5 M NaCl, pH 4).

9. The beads were washed three times with coupling buffer (0.5 M ethanolamine, 0.5 M NaCl, pH 8.3).

10. The beads were incubated with 500 μl of blocking buffer for one hour with rotation at room temperature to allow for deactivation or blocking of any leftover active groups.

11. The beads were washed with (a) coupling buffer and then with (b) acetate buffer. This wash ((a) then (b)) was repeated three times.

12. The beads were washed two times in 1× annealing buffer. The annealing buffer also serves as the storage buffer.

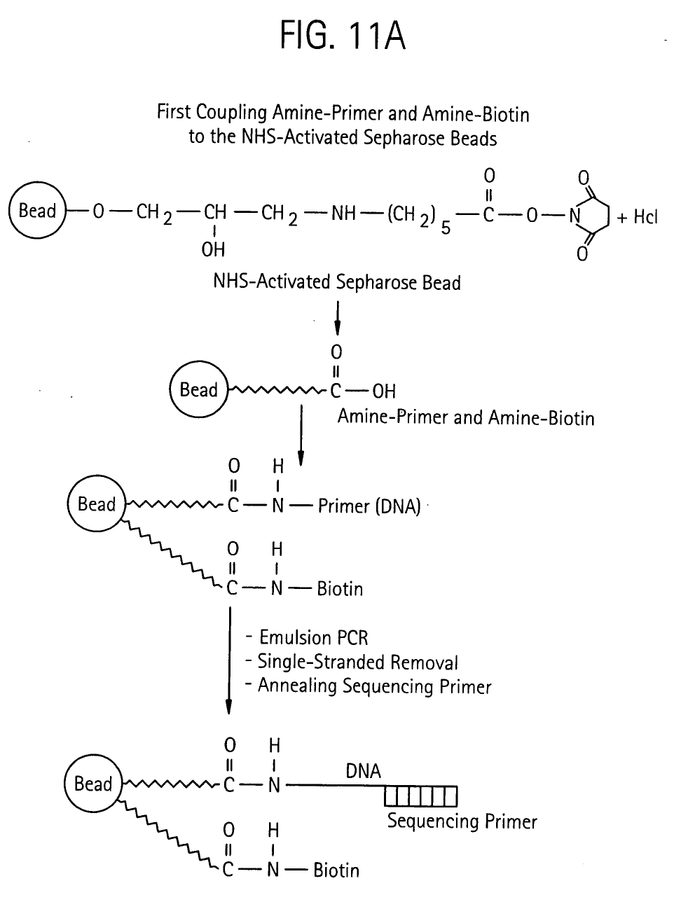

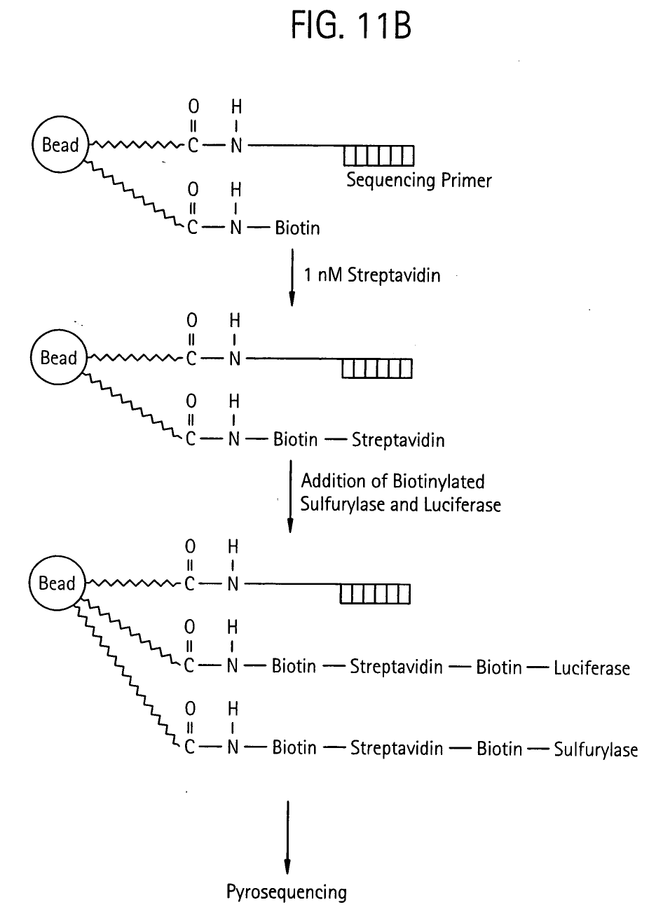

This procedure is illustrated in FIG. 11.

In a particularly preferred embodiment, the beads and bioactive agents are linked using a biotin/streptavidin linkage, well known to those skilled in the art.

Apparatus for Detecting a Reaction in the Membrane Reactor Array:

The invention provides an apparatus for simultaneously monitoring an array of reaction chambers for light indicating that a reaction is taking place at a particular site. The reaction event, e.g., photons generated by luciferase, may be detected and quantified using a variety of detection apparati, e.g., a photomultiplier tube, a CCD, CMOS, absorbance photometer, a luminometer, charge injection device (CID), or other solid state detector, as well as the apparati described herein. In a preferred embodiment, the quantitation of the emitted photons is accomplished by the use of a CCD camera fitted with a fused fiber optic bundle. In another preferred embodiment, the quantitation of the emitted photons is accomplished by the use of a CCD camera fitted with a microchannel plate intensifier. A back-thinned CCD can be used to increase sensitivity. CCD detectors are described in, e.g., Bronks, et al., 1995. Anal. Chem. 65: 2750-2757. The CCD sensitivity may be enhanced by the known method of chilling the CCD during exposure.

An exemplary CCD system is a Spectral Instruments, Inc. (Tucson, Ariz.) Series 600 4-port camera with a Lockheed-Martin LM485 CCD chip and a 1-1 fiber optic connector (bundle) with 6-8 μm individual fiber diameters. This system has 4096×4096, or greater than 16 million pixels and has a quantum efficiency ranging from 10% to >40%. Thus, depending on wavelength, as much as 40% of the photons imaged onto the CCD sensor are converted to detectable electrons.

The invention also provides a microimaging system for imaging a light emission (e.g., from a pyrophosphate sequencing reaction) from a membrane reactor array. The system comprises two lens groups. The first lens group is the front lens group which is positioned closer to the light source to be detected to collect the light emitted. The second lens group is the rear lens group that is positioned closer to the light detector such as a CCD detection device to image the light onto the detector. In one aspect of the invention, the front lens group and rear lens group are identical.

In a preferred embodiment, the lens group comprises 50 mm lens with an aperture larger than 2.8 (e.g., 2.0, 1.8, 1.4, 1.0, etc.). Preferably, a lens group has a focal length of at least 30 mm, at least 50 mm, or at least 70 mm. In specific aspects, a lens group has an aperture brighter than or equal to 4.0, or brighter than or equal to 2.8. In other aspects, a lens group has a numerical aperture larger than 0.1, 0.2, or 0.3. It should be noted that the larger apertures are expressed by a smaller aperture value so that, for example, an aperture of 1 is larger than an aperture of 2. An exemplary imaging system is shown in FIG. 2.

The data from the optical detection device can be analyzed instantaneously or stored electronically (e.g., by computers, hard drives, optical drives, solid state memories) for subsequent analysis as is known to those of skill in the art.

In other embodiments, a fluorescent moiety can be used as a label and the detection of a reaction event can be carried out using a confocal scanning microscope to scan the surface of an array with a laser or other techniques such as scanning near-field optical microscopy (SNOM) which are capable of smaller optical resolution, thereby allowing the use of “more dense” arrays. For example, using SNOM, individual polynucleotides may be distinguished when separated by a distance of less than 100 nm, e.g., 10 nm×10 nm. Additionally, scanning tunneling microscopy (Binning et al., Helvetica Physica Acta, 55:726-735, 1982) and atomic force microscopy (Hanswa et al., Annu Rev Biophys Biomol Struct, 23:115-139, 1994) can be used.

Additional material may be found in U.S. application Ser. No. 10/191,438 filed Jul. 8, 2002, U.S. Application Ser. No. 60/476,592 filed Jun. 6, 2003, U.S. Application Ser. No. 60/476,602, filed Jun. 6, 2003, U.S. Application Ser. No. 60/476,313 filed Jun. 6, 2003, U.S. Application Ser. No. 60/476,504 filed Jun. 6, 2003, and U.S. Pat. 6,274,320. All patent applications and patents, listed in this disclosure, are hereby incorporated by reference in their entirety.

Many variations and alternative embodiments of the present invention as applied to DNA sequencing and other applications will be readily apparent and are considered to be within the scope of the present invention.

### EXAMPLES

### Example 1

### Sequencing UATF9 DNA Template On Convective Rig

Preparing the Beads

Streptavidin-sepharose beads were size-selected by filtering to obtain diameter between 30-36 μm. The primers and target DNA included: MMP7A sequencing primer (5′-ccatctgttc cctccctgtc-3′; SEQ ID NO:6); target DNA, termed UATF9 (3′-ATGCCGCAAA AACGCAAAAC GCAAACGCAA CGCATACCTC TCCGCGTAGG CGCTCGTTGG TCCAGCAGAG GCGGCCGCCC TTGCGCGAGC AGAATGGCGG TAGGGGGTCT AGCTGCGTCT CGTCCGGGG-5′; SEQ ID NO:7); biotinylated primer and PCR reverse primer, termed Bio-Heg-MMP1B (5′-5Bio//iSp18//iSp18//iSp18/cca tct gtt gcg tgc gtg ct-3′; SEQ ID NO:8); and PCR forward primer, termed MMP1A (5′-cgtttcccct gtgtgccttg-3′; SEQ ID NO:9). For the PCR reverse primer, “5Bio” indicates biotin and “iSp18” indicates Spacer 18.

The biotinylated PCR products were immobilized onto Streptavidin-Sepharose beads. Immobilized PCR product was incubated in 0.10 M NaOH for 10 min, and the supernatant was removed to obtain single-stranded DNA. The beads containing single-stranded DNA were washed 3 times with 100 μl of 1× Annealing Buffer, pH 7.5 (30 mM Tris-HCl, 3 mM magnesium acetate, from Fisher). The beads were pelleted by centrifugation for 1 min at a maximum speed of 13,000 rpm. Supernatant was removed and the beads were suspended in 25 μl of 1× Annealing Buffer. Five microliter of 100 μmol sequencing primer was added to mixture. The beads were incubated at 65° C. for 5 min and cooled to room temperature. The beads were washed 3 times with 100 μl of 1× Annealing Buffer and resuspended in final volume of 100 μl.

Loading the Beads Into The Membrane and Assembly Into the Loading Jig

The beads were resuspended at a concentration of about 3,500 beads per microliter in 1× Annealing Buffer. Following this, 25 μl of the suspension was added to 200 μl of 1× Assay Buffer, pH 7.8 (25 mM tricine (Fisher), 5 mM magnesium acetate (Fisher), 1 mM dithiothreitol, 0.4 mg/ml polyvinylpyrrolidone, 0.01% Tween 20, 1 mg/ml BSA, all from Sigma (St. Louis, Mo.)). In the loading jig, a 0.2 μm nylon membrane was placed below the 30 μm pore nylon membrane (Sefar). The loading jig was then attached to a peristaltic pump having a flow rate of 1 ml per min. Next, 200 μl of 1× Assay Buffer was used to wash the membrane. After washing, and while the membrane was partially dry, 200 μl of the bead suspension was added to the membrane. Negative pressure was applied to the membrane along with 500 μl of 1× Assay Buffer to force the beads onto the pores of the membrane.

After the application of beads, the membrane was washed with 500 μl of 1× Assay Buffer. The membrane was disassembled and placed in 1× Assay Buffer (e.g., 20 ml) in a test tube (e.g., Falcon) for storage. The following solutions were mixed in a container: 500 μl of biotinylated ATP Sulfurylase enzyme at 1 mg/ml (454 Life Sciences); 500 μl of biotinylated Luciferase at 3 mg/ml (454 Life Sciences); 500 μl of 1× Assay Buffer. The bead-loaded nylon membrane was placed in the enzyme mixture. The mixture was rotated with the nylon membrane for 20 min at room temperature at a speed of one rotation per two seconds. The membrane was then placed in 20 ml of 1× Assay Buffer and swirled for 2 minutes. This wash step was repeated once. A solution of 970 μl 1× Assay Buffer with 30 μl of Bst Polymerase enzyme (New England Bio Labs) was prepared. The membrane was immersed in this solution, and the solution/membrane was rotated for 25 min at room temperature at a speed of one rotation per two seconds. After the rotations, the membrane was washed two times in 20 ml of 1× Assay buffer.

Preparing the Membrane for Sequencing on Convective Rig:

A 0.2 μm nylon membrane was immersed in 20 ml of 1× Assay Buffer and placed above the wire mesh on the sequencing Jig holder. A 30 μm membrane containing the DNA beads was placed on top of the 0.2 μm nylon membrane. A sequence Jig cover with an optical glass window (13 mm) was placed on top of the membranes. The Jig cover was tightly attached to the lower part of the membrane holder. The cover and holder were threaded and tightening was performed by screwing the two parts together. The sequencing Jig was placed on the z-translation stage below the CCD camera. The acquisition time on the camera was set to 7 sec and the read out time was set to 0.25 sec. The inlet of the membrane holder was connected to the outlet of the pump, which was connected to the Valco valve (Valco Instruments, Houston, Tex.). The lower part of the membrane holder was connected to the second peristaltic pump. The outlet of the sequencing chamber was connected to the waste.

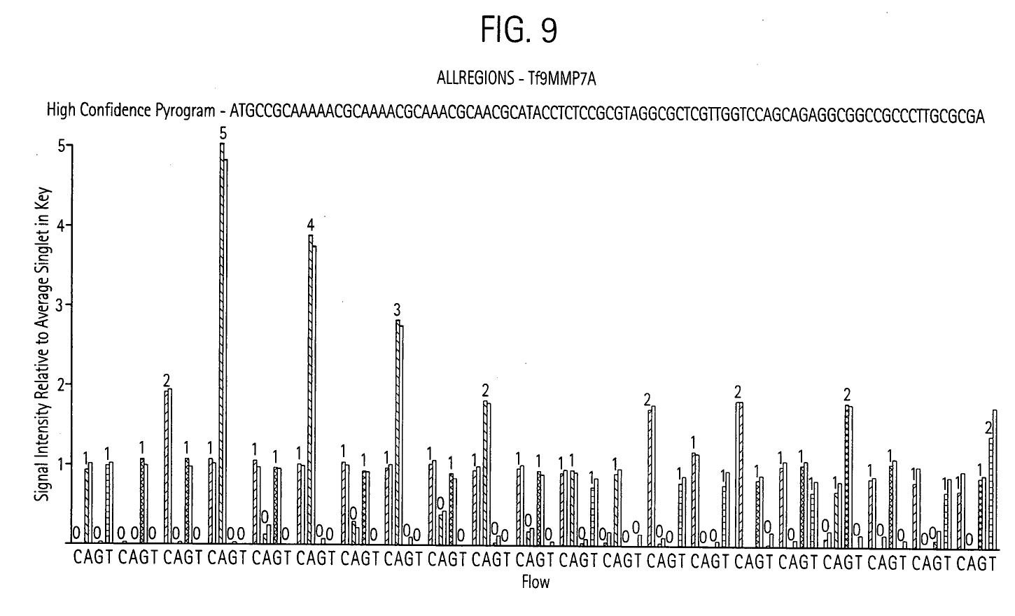

The substrate (25 mM tricine, 5 mM magnesium acetate, both from Fisher; 1 mM dithiothreitol, 0.4 mg/ml polyvinylpyrrolidone, 0.01% Tween 20, all from Sigma; 300 μM D-Luciferin, from Regis; 2.5 μM adenosine-5′-O-phosphosulfate, from Axxora, Inc.) was flowed for 2 min to prime the flow chamber and expel any air bubbles. This allowed the DNA on the nylon membrane to be equilibrated with substrate. The reagents were flowed through the chamber in the following order: 1) dCTP; 2) substrate; 3) Nuke Sp-dATP-α-thio; 4) substrate; 5) dGTP; 6) substrate; 7) dTTP; and 8) substrate. This cycle was repeated 20 times. The flow rates for the lateral and vertical flow were controlled as follows. The nucleotide flow (for steps (1), (3), (5), and (7), above) was at 2 ml/min lateral for 21 sec and then 0.5 ml/min for 7 sec. The vertical flow was 0.5 ml/min for the same time. Total time was 28 sec. The substrate flow (for steps (2), (4), (6), and (8), above) was at 2 ml/min lateral flow and 1 ml/min vertical flow 1 ml/min for 77 sec. The results of the sequencing reaction are shown in FIG. 9.

### Example 2

### PCR on Nylon Membrane Containing Beads and Sequencing Using a Pyrosequencer: The Sequencing Step was to Confirm the Fidelity of the Amplified Template

The primers and probe included:

| Name | Sequence | SEQ ID NO: |
|---|---|---|
| Adeno P1 forward | 5′ caa tta acc ctc act aaa gg 3′ | 1 |
| Adeno P2 reverse | 5′ gta ata cga ctc act ata ggg 3′ | 2 |
| tf2 | 3′cgatcaagcgtacgcacgtggttgttaaagcttttttgaaagttaatc | 3 |
| | tcctggttcaccgtctgctcgtatgcggttaccaggtcggcggccgcc | |
| | acgtgtgcgcgcgcgggactaatcccggttcgcgcgtcgg 5′ | |
| Biotinylated probe Adeno P1 | 5′/Bio//iSp18//iSp18/iSp18/caa tta acc ctc act aaa | 4 |
| | gg 3′ | |

The sepharose beads were treated as in Example 1, with a concentration of 3,500 beads per microliter. Next, 90 μl of sepharose beads were washed by resuspension in 200 μl of 1× PCR buffer and this was followed by centrifugation for a total of three washes. After the final wash, 200 μl of 1× PCR buffer was placed on top of the beads pelleted by centrifugation. Then, 6 μl of 100 pmol/μl biotinylated P1 probe was added to the top of the beads/PCR buffer. The beads were resuspended and the tube containing the beads was placed on a rotator for 45 min at room temperature. Following the rotation, the beads were washed three times with 200 μl 1× PCR buffer. A 15 μl aliquot of bead suspension was placed in a microcentrifuge tube and briefly centrifuged for 30 s at 13.2 k rpm to let the beads settle down. This sample was marked “Sample A”. Another sample, “Sample B” was prepared the same way.

The aqueous layer above the beads in Sample A (negative control) was removed and replaced with 50 μl of PCR mix (37.7 μl H₂0, 5 μl 10×PCR buffer, 1 μl dNTPs (10 mM each), 0.4 μl of 100 pmol/μl forward primer, 0.4 μl of 100 pmol/μl P2 reverse primer, 5 μl Betaine (5 M), and 0.5 μl of Taq polymerase (5 U/μl). Nylon membranes were cut into 2 mm circles using a die cutter. The circles were pre-wetted by immersion in 1×PCR buffer. One 2 mm circle of nylon membrane was immersed in Sample A such that the beads were attached to the membrane; filling most of the pores. Sample A and the nylon membrane were placed on a rotator for 3 hours at 4° C. to allow the beads to take up the components of the PCR mixture. The nylon membrane was removed and fully immersed in a tube containing about 20 μl of mineral oil.

The aqueous layer above the beads in Sample B was replaced with 50 μl PCR mix (35.45 μl H₂O, 5 μl 10×PCR buffer, 1 μl dNTPs (10 mM each), 0.4 μl of 100 pmol/μl P1 forward primer, 0.4 μl of 100 pmol/μl P2 reverse primer, 5 μl Betaine (5 M), 0.5 μl Taq polymerase (5 U/μl) and 2.25 μl of 1.67 attomol/μl tf2 adeno fragment. One attomole is defined as 1×10⁻¹⁸ moles. The estimated concentration of DNA per bead prior to amplification was 10 copies per bead. (Note that where each bead was to contain a distinct template sequence, then a maximum of one unique sequence per bead was preferred). One 2 mm circle of nylon membrane was immersed in Sample B such that the beads were attached to the membrane; filling most of the pores. Sample B and the nylon membrane were placed on a rotator for 3 hours at 4° C. to allow the beads to take up the components of the PCR mixture. The nylon membrane was removed and fully immersed in a tube containing about 20 μl of mineral oil.

The tube containing the nylon membrane with Sample A and the tube containing the nylon membrane with Sample B were placed in a thermocycler with the following reaction conditions. Step 1: 96 C for 2 min; Step 2: 96 C for 1 min; Step 3: 58 C for 1 min; Step 4: 72 C for 1 min, go to step 2, 29 times; Step5: 72 C for 10 min; Step 6: 14 C overnight or until the reaction was terminated. After the PCR reaction, the PCR tubes with the nylon membranes were removed from the thermocycler. The membranes were removed and placed into separate tubes containing 1 ml chloroform and 200 μl of 1× Annealing Buffer. The tubes were shaken several times. The membranes were transferred to individual tubes containing 1 ml of chloroform and the tubes were rotated several times. The membranes were then transferred to individual tubes with 200 μl of 1× Annealing Buffer. The tubes were then rotated several times. This procedure was repeated an additional two times with 200 μl of Annealing Buffer.

To denature DNA on the beads, the membranes were transferred to individual tubes with 50 μl of 1× Annealing Buffer. The tubes were heated to 90 C for 2 min in a PCR thermocycler. Next, the membranes were transferred to individual tubes containing 50 μl of 1× Annealing Buffer on ice. The membranes were washed two times with 100 μl of 1× Annealing Buffer. The membranes were then incubated with a solution of 5 μl of 100 pmol of P2 primer mixed with 20 μl of 1× Annealing Buffer. The tubes were placed in a thermocycler and heated to 65° C. Next, the tubes were slowly cooled to room temperature to allow the P2 sequencing primer to anneal to the DNA template. Following annealing, the membranes were washed two times with 100 μl of 1× Annealing Buffer.

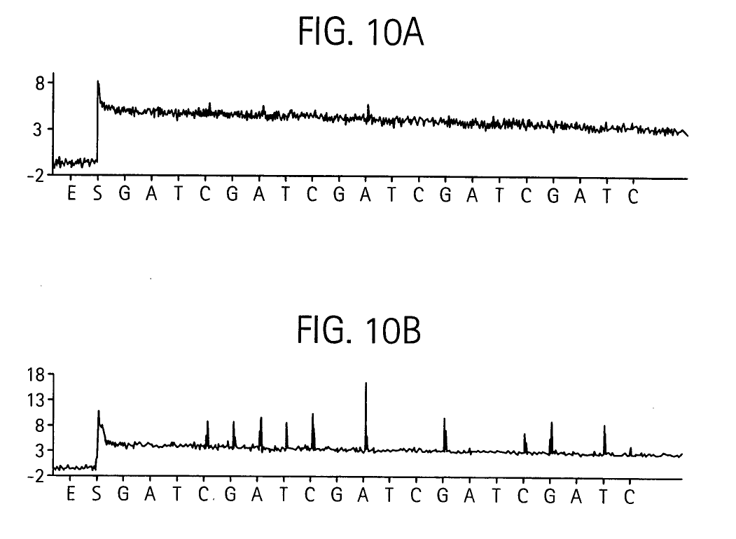

To confirm the fidelity of the amplified DNA fragment, the reaction product was sequenced on the beads using a pyrophosphate sequencer (PSQ). Methods of pyrophosphate sequencing are generally described, e.g., in U.S. Pat. Nos. 6,274,320, 6258,568 and 6,210,891, incorporated in toto herein by reference. Briefly, the membranes were soaked for 30 sec in 50 μl of a mixture of ATP sulfurylase and luciferase enzymes (454 life Sciences). Then, the membrane was placed into a well of the PSQ. The nucleotides were flowed into the PSQ plates in the order of G, A, C, and then T. This was repeated five times. For the sample with no DNA (the negative control), no sequence was detected (FIG. 10A). For the amplified tf2 fragment on the beads on the membrane, the proper sequence was detected (FIG. 10B). Thus, detectable sequence was obtained starting from a small number of DNA template fragments (10 copies per bead), using the amplification and sequencing reactions described herein.

### Example 3

### Methods for Pyrosequencing

Any DNA may be sequenced using the procedure described herein. Briefly, beads are filtered to obtain a diameter of 25-30 μm and resuspended at a concentration of 3,500 beads/μl, as described above. Next, 14 μl of the bead solution is placed into a tube for each sample to be sequenced. The beads are pelleted at 13,000 rpm. The supernatant is replaced with 500 μl of a mixture of the three enzymes (6 μl of sulfurylase at 1 mg/ml, 6 μl of luciferase at 3 mg/ml, and 60 μl of Bst polymerase at 50 U/μl) and 428 μl of 1× Assay Buffer containing 1 mg/ml BSA. The tube is placed in a rotator for 1 hr at room temperature, at about one turn every 2 sec. Then, the beads are pelleted by centrifugation at 2,000 rpm for 2.5 min. The beads are washed once with 200 μl of 1× Assay Buffer without BSA. Then the beads are loaded onto a membrane with 30 μm pore for pyrophosphate sequencing.

### Example 4

### Bead Loading Methods

A membrane (e.g., nitrocellulose membrane circle, as described herein) is dipped into bead solution such as 1× Assay Buffer. The membrane is agitated to trap beads in the membrane pores. The membrane/bead mixture is submerged in bead solution. This is stirred or vortexed to trap the beads in the membrane pores. The membrane is used as a bead filter in a sieve, and the bead solution is drained through the membrane using gravity or centrifugal force. On an open-loading Jig and a centered membrane, the bead solution is introduced from the top and drained through the bottom by a pump. Mixing can be used in the cavity of the Jig to ensure uniform bead distribution on the membrane. The loading Jig can include multiple cavities for bead deposits onto different areas of the membrane for different samples or tests. This method can be combined with the loading Jig method as described herein. The beads are loaded using a wicking effect. The membrane is placed on top of other highly hydrophilic membrane or tissue, or other wicking material, and the bead solution is applied to the membrane. Using a pump, the bead solution is flowed across the membrane in an enclosed chamber. The chamber can be placed in any orientation, and the beads can be introduced by various-means such as a syringe, pipette, duct, tubing, and the like. The arrayed sample delivery devices (manual or automated) can be used to deposit beads onto discrete regions or patches on the membrane.

### Example 5

### Automated Convective Sequencing Protocol

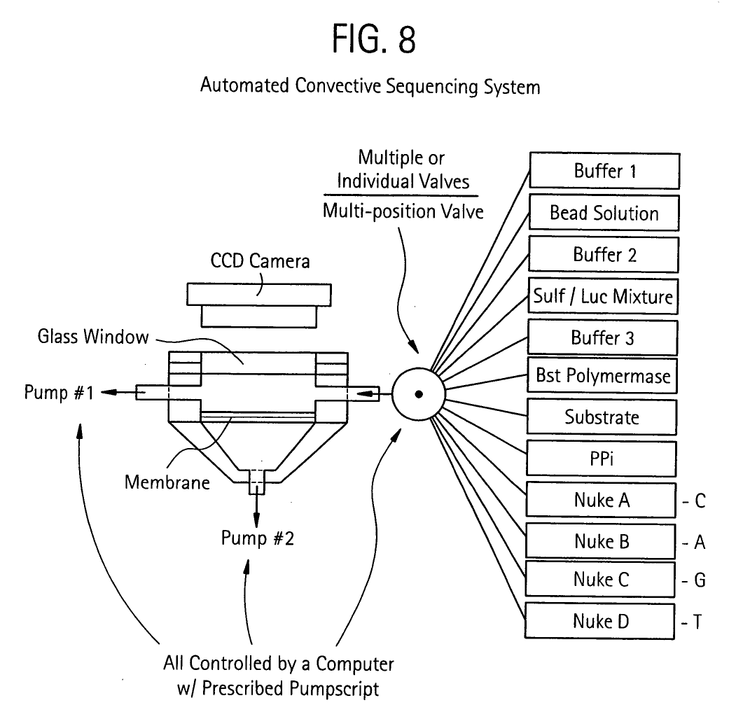

Wash buffers, sample DNA, bead solution, enzyme solutions, and sequencing reagents (substrate, PPᵢ, and nucleotides) are prepared according to the layout of the automated sequencing system (FIG. 8). The sequencing and resistance membranes are incubated in 1× Assay Buffer (AB) containing 1% bovine serum albumin (BSA) for at least 15 min, preferably 30 min, to prevent PPᵢ drop during a long sequencing run. The sequence chamber system is assembled with the membranes sand connected to pumps and reagents. The beads are loaded with Pump 2 at a flow rate of 1-2 ml/min depending on the chamber size. The chamber is washed with a wash buffer (1× AB with 1% BSA). Non-binding beads are removed by running Pump 2 while switching to Pump 1. A proper flow rate is set so that it removes loose beads without disrupting the membrane.

A sulfurylase and luciferase mixture is loaded with both Pump 1 and Pump 2 running. This is incubated for 15 min with mixing by the reciprocal movement by Pump 2. The chamber is washed with a wash buffer for polymerase with both pumps running for 5 min. A Bst polymerase (New England Biolabs, Beverly, Mass.) solution is loaded and incubated for 30 min with mixing. The chamber is washed with substrate for 5 min with both pumps running. Alternatively, the Bst polymerase can be mixed with sulfurylase and luciferase for combined infusion with all three enzymes. A 0.1 μM PPᵢ solution is run for signal calibration with a flow rate of 1.5 ml/min for both pumps for 21 sec. After this, Pump 1 is stopped and Pump 2 is continued for another 14 sec. The substrate is washed for 115 sec with Pump 1 at 1 ml/min and Pump 2 at 2 ml/min. The nucleotides are added in order (e.g., C, A, G, T) for a predetermined number of cycles with the same pump procedure as used for PPᵢ, except that a 65 sec substrate wash is used after each nucleotide is added. At the end of the nucleotide runs, PPᵢ is run again to check if enzyme activity changes with time. The data is analyzed using DNA sequencing software.

Additional methods that may be used with the invention can be found in U.S. application Ser. No. 10/191,438 filed Jul. 8, 2002, U.S. Application Ser. No. 60/476,592 filed Jun. 6, 2003, U.S. Application Ser. No. 60/476,602, filed Jun. 6, 2003, U.S. Application Ser. No. 60/476,313 filed Jun. 6, 2003, U.S. Application Ser. No. 60/476,504 filed Jun. 6, 2003, and U.S. Pat. No. 6,274,320, which are hereby incorporated by reference herein in their entirety.

### REFERENCES

Anderson J R, Chiu D T, Jackman R J, Cherniavskaya O, McDonald J C, Wu H, Whitesides S H, and Whitesides G M. 2000. Fabrication of topologically complex three-dimensional microfluidic systems in PDMS by rapid prototyping. Anal. Chem. 72:3158-64.

Asoh, H, Nishio, K, Nakao, M. Tamamura, T and Masuda, H. 2001. Conditions for fabrication of ideally ordered anodic porous alumina using pretextured aluminum. J. Electrochem. Soc. 148:B152-B156.

Cherukuri S C, Demers R R, Fan Z H H, Levine A W, McBride S E, Zanzucchi P J. 1999. Method and system for inhibiting cross-contamination in fluids of combinatorial chemistry device. U.S. Pat. No. 5,980,704.

Dannoux T, Pujol G, and Root D. 2000. High-density test plate and process of making. U.S. Pat. No. 6,030,829.

Deng T, Wu H, Brittain S T, and Whitesides G M. 2000. Prototyping of masks, masters, and stamps/molds for soft lithography using an office printer and photographic reduction. Anal. Chem. 72:3176-80.

Ehrfeld, W, Hessel, V, and Lowe, H. 2000. Microreactors: New Technology for Modern Chemistry. New York: Wiley-VCH.

Eykamp, W. 1995. Microfiltration and ultrafiltration, pp. 1-43 in Membrane Separation Technology: Principles and Applications, R D Noble and S A Stem, eds. Amsterdam: Elsevier Science BV.

Furneaux, R C, Rigby, W R, and Davidson, A P. 1989. The formation of controlled porosity membranes from anodically oxidized aluminum. Nature. 337:147-9.

Hyman E D. 1988. A new method for sequencing DNA. Anal. Biochem. 174:423-36.

Jansson V and Jansson K. 2002. Enzymatic chemiluminescence assay for inorganic pyrophosphate. Analytical Biochemistry. 304:135-137.

Jensen N. 2002. Flow-through processing on a microchip for DNA pyrosequencing. Natl. Nanofabrication Users Network. 66-67.

Kane R S, Takayama S, Ostuni E, Ingber D E, and Whitesides G M. 1999. Patterning proteins and cells using soft lithography. Biomaterials. 20:2363-76.

Kulkarni, S S, Funk, E W, and Li, N N. 1992. Ultrafiltration, pp. 391-453 in Membrane Handbook, WSW Ho and K K Sirkar, eds. New York: Van Nostrand Reinhold.

Mardilovich, P P, Govyadinov, A N, Mukhurov, N I, Rzhevskii, A M, and Paterson, R.

New and modified anodic alumina membranes. Part I: Treatment of anodic alumina membranes. Part II: Comparison of solubility of amorphous (normal) and polycrystalline anodic alumina membranes. J. Memb. Sci. 98:131-155.

Martin C R. 1994. Nanomaterials—A membrane-based synthetic approach. Science. 266:1961-1966.

Matsuda T and Chung D J. 1994. Microfabricated surface designs for cell culture and diagnosis. ASAIO J. 1 :M594-7.

Michael K L, Taylor L C, Schultz S L, and Walt D R. 1998. Randomly ordered addressable high-density optical sensor arrays. Anal. Chem. 70:1242-8.

Nyren P, Pettersson B, and Uhlén M. 1993. Solid phase DNA minisequencing by an enzymatic luminometric inorganic pyrophosphate detection assay. Anal. Biochem. 208:171-5.

Rai-Choudhury P. 1997. Handbook of Microlithography, Micromachining and Microfabrication. Vol. 1: Microlithography and Vol. 2: Micromachining and Microfabrication. SPIE Press.

Ronaghi M, Uhlén M, and Nyr\acute{en}P. 1998. A sequencing method based on real-time pyrophosphate. Science. 281:363-5.

Schuller J. 2002. Microfluidics for DNA pyrosequencing. Natl. Nanofabrication Users Network. 76-77.

Taylor L C and Walt D R. 2000. Application of high-density optical microwell arrays in a live-cell biosensing system. Anal. Biochem. 278:132-42

Zhu H, Klemic J F, Chang S, Bertone P, Casamayor A, Klemic K G, Smith D, Gerstein M, Reed M A, and & Snyder M. 2000. Analysis of yeast protein kinases using protein chips. Nature Genet. 26:283-89.

We Claim:

1. A membrane reactor array comprising:
a planar array layer comprising a plurality of pores with reagent-carrying beads disposed in a plurality of said pores, with a maximum of one bead per pore, wherein the array is substantially permeable to aqueous solutions but is substantially impermeable to said beads; and
a porous high flow resistance membrane layer, in contact with the planar array layer, that is permeable to nucleic acids and proteins to provide flow resistance of greater than 10 fold that of the flow resistance of the aqueous solutions through a bead-containing pore in said planar array.

2. The membrane reactor array of claim 1 wherein said planar array layer is formed to provide a spacing of pores of less than 100 μm center to center.

3. The membrane reactor array of claim 1 wherein said planar array layer is formed to provide a spacing of pores of about 50 to 78 μm center to center.

4. The membrane reactor array of claim 1 wherein said planar array layer comprises pores with a pore size length in one dimension of about 15 μm to 100 μm in diameter.

5. The membrane reactor array of claim 1 wherein said planar array layer comprises pores of a pore size length in one dimension of about 20 to 35 μm in diameter.

6. The membrane reactor array of claim 1 wherein said planar array layer comprises substantially square pores with an average size of about 15 to 100 μm.

7. The membrane reactor array of claim 1 wherein said planar array layer comprises substantially square pores with an average size of about 20 to 35 μm.

8. The membrane reactor array of claim 1 wherein said planar array layer comprises substantially square pores with an average size of about, 25 μm, 28 μm, 30 μm or 31 μm.

9. The membrane reactor array of claim 1 wherein said planar array layer comprises pores that are rectangular.

10. The membrane reactor array of claim 1 wherein said planar array layer comprises at least 10,000 pores.

11. The membrane reactor array of claim 1 wherein said planar array layer comprises at least 50,000 pores.

12. The membrane reactor array of claim 1 wherein said planar array layer comprises at least 100,000 pores.

13. The membrane reactor array of claim 1 wherein said planar array layer comprises at least 250,000 pores.

14. The membrane reactor array of claim 1 wherein said high flow resistance membrane has a pore size between 0.01 μm and 10 μm.

15. The membrane reactor array of claim 1 wherein said high flow resistance membrane has a pore size between 0.01 μm and 0.5 μm.

16. The membrane reactor array of claim 1 wherein said high flow resistance membrane has a pore size between less than 0.1 μm and 1 μm.

17. The membrane reactor array of claim 1 wherein said high flow resistance membrane is a hydrophobic membrane with a pore size less than 20 μm.

18. The membrane reactor array of claim 1 wherein said high flow resistance membrane has a flow resistance to a liquid that is 100 fold greater than that of the planar array layer.

19. The membrane reactor array of claim 1 wherein said high flow resistance membrane has a pore diameter that is less than 10% of the pore diameter of the planar array layer.

20. The membrane reactor array of claim 1 wherein said high flow resistance membrane has a pore diameter that is less than 1% of the pore diameter of the planar array layer.

21. The membrane reactor array of claim 1 wherein said planar array layer is a woven mesh of a plurality of fibers with pores formed between said fibers.

22. The membrane reactor array of claim 1 wherein said planar array layer is a planar surface comprising pores.

23. The membrane reactor array of claim 1, additionally comprising a porous structural support layer which has less resistance to fluid flow than the planar array or the porous high flow resistance membrane layer.

24. The membrane reactor array of claim 23, wherein said porous structural support layer is made from a material selected from the group consisting of metal, ceramic, and porous silicon.

25. The membrane reactor array of claim 1, further comprising a pyrophosphate degrading enzyme immobilized on one said layer or on said bead.

26. A cartridge comprising a membrane reactor array of claim 1 wherein said membrane reactor array bisects said cartridge into a first and a second chamber, and wherein said cartridge comprise a first opening in said first chamber and a second opening in said second chamber to allow a fluid to flow through said first opening, through said membrane reactor array, and through said second opening.

27. The cartridge of claim 26, further comprising a third opening in said first chamber to allow a fluid to flow through said first opening laterally over said membrane reactor array and through said third opening.

28. A membrane reactor array, comprising
(a) a planar array layer comprising a plurality of pores with reagent-carrying beads disposed in a plurality of said pores such that a maximum of one bead is disposed within any one pore, wherein said beads restrict but do not completely block the flow of fluid through said pores, and wherein the array is substantially permeable to aqueous solutions but is substantially impermeable to said beads, and wherein said planar array is made from a fiber material of substantially uniform cross-section weaved to form a mesh layer comprising an array of pores of uniform size, and wherein each pore is an opening of less than 40 μm; and
(b) a porous high flow resistance membrane layer, in contact with the planar array layer, that is permeable to nucleic acids and proteins to provide flow resistance of greater than 10 fold that of the flow resistance of aqueous solutes through a bead containing pore in said planar array.

29. The membrane reactor array of claim 28 wherein said planar array layer comprises two sets of parallel fibers woven together such that the two sets of parallel fibers are substantially at right angle to each other, wherein each individual fiber of each set has a center to center spacing of less than 100 μm, and wherein the fibers form an array of pores.

30. The membrane reactor array of claim 28 wherein said planar array layer comprises pores with a pore size length in one dimension of about 20 μm to 35 μm and a spacing of pores of about 50 to 78 μm center to center.

31. The membrane reactor array of claim 28 wherein the percentage of pores in the mesh layer that are loaded with beads is selected from the group consisting of 5%, 10%, and 25%.

32. The membrane reactor array of claim 28 wherein at least one sequencing enzyme is immobilized on said mesh layer or said bead.

33. The membrane reactor array of claim 32 wherein the sequencing enzyme is selected from the group consisting of ATP sulfurylase, luciferase, polymerase, hypoxanthine phosphoribosyltransferase, xanthine oxidase, uricase, and peroxidase.

34. The membrane reactor array of claim 28 wherein said array is substantially impermeable to a bead with a diameter of more than 40 μm.

35. The membrane reactor array of claim 28 wherein said plurality of beads are attached to at least one nucleic acid molecule.

36. The membrane reactor array of claim 35 wherein each said bead is attached to a single nucleic acid molecule.

37. The membrane reactor array of claim 35 wherein each said bead is attached to a single species of nucleic acid molecule.

38. The membrane reactor array of claim 32, further comprising a pyrophosphate degrading enzyme immobilized on one said layer or on said bead.

39. A method of identifying a base at a target position in a sample DNA sequence comprising:
providing a sample DNA sequence and an extension primer that hybridizes to the sample DNA immediately adjacent to the target position and subjecting the sample DNA and extension primer to a polymerase reaction in the presence of a deoxynucleotide or deoxynucleotide,
wherein said sample DNA sequence is immobilized on a bead within a membrane reactor array of claim 1; and
whereby the deoxynucleotide or dideoxynucleotide will only become incorporated and release pyrophosphate (PPᵢ) if it is complementary to the base in the target position, and any release of PPᵢ can be detected enzymatically;
adding different deoxynucleotides or dideoxynucleotides successively to the ample-primer mixture; and
determining which deoxynucleotide or dideoxynucleotide is incorporated, thereby identifying a base that is complementary to the base at the target position.

40. The method of claim 39 wherein said different deoxynucleotides or dideoxynucleotides are added to said DNA sequence by a fluid flow that is normal to a plane of the planar membrane reactor array.

41. The membrane reactor array of claim 40 wherein said fluid flow has a flow rate of 0.15 ml/minute/cm² to 4 ml/minute/cm².

42. A method of identifying a base at a target position in a sample DNA sequence comprising:
arranging a multiplicity of DNA sequences, each immobilized on a bead, in array format on a membrane reactor array of claim 1;
providing to each sample an extension primer, which hybridizes to the sample DNA immediately adjacent to the target position and subjecting the sample DNA and extension primer to a polymerase reaction in the presence of a deoxynucleotide or dideoxynucleotide, whereby the deoxynucleotide or dideoxynucleotide will only become incorporated and release pyrophosphate (PPᵢ) if it is complementary to the base in the target position and detecting any release of PPᵢ enzymatically, and whereby a nucleotide-degrading enzyme is included during the polymerase reaction such that unincorporated nucleotides are degraded;
adding different deoxynucleotides or dideoxynucleotides successively to the same sample-primer mixture in a fluid flow that is normal to the plane of the planar mesh;
determining which deoxynucleotide or dideoxynucleotide is incorporated, thereby identifying a base that is complementary to the base at the target position.

43. The method of claim 42 wherein said nucleotide-degrading enzyme is apyrase.

44. A method of loading a membrane reactor array comprising the steps of:
(a) providing a membrane reactor array of claim 1 which is substantially permeable to a fluid but substantially impermeable to a population of microreactor beads;
(b) depositing said fluid comprising a suspension of said population of microreactor beads on the surface of the membrane reactor array; and
(c) settling said microreactor beads onto the pores at no more than one bead per pore.

45. The method of claim 44 wherein said settling comprises drawing said fluid through said membrane reactor array whereby said microreactor beads are trapped in said membrane reactor array.

46. The method of claim 44 wherein said microreactor beads have a diameter of between 20 to 35 μm.

47. A microimaging lens system for imaging a light emission from a membrane reactor array of claim 1 comprising:
(a) a front lens group for collecting a light emission from a sequencing reaction; and
(b) a rear lens group for imaging said light emission in a spatially ordered manner onto an optical detector.

48. The microimaging lens system of claim 47 wherein at least one said lens group has a focal length of at least 30 mm focal length.

49. The microimaging lens system of claim 47 wherein at least one said lens group has a focal length of at least 50 mm focal length.

50. The microimaging lens system of claim 47 wherein at least one said lens group has a focal length of at least 70 mm focal length.

51. The microimaging lens system of claim 47 wherein at least one said lens group has an aperture brighter than or equal to 4.0.

52. The microimaging lens system of claim 47 wherein at least one said lens group has an aperture brighter than or equal to 2.8.

53. The microimaging lens system of claim 47 wherein at least one said lens group has a numeric aperture larger than or equal to 0.1.

54. The microimaging lens system of claim 47 wherein at least one said lens group has a numeric aperture larger than or equal to 0.2.

55. The microimaging lens system of claim 47 wherein at least one said lens group has a numeric aperture larger than or equal to 0.3.

56. The microimaging lens system of claim 47 wherein said front lens group and rear lens group are identical.

57. The microimaging lens system of claim 47, further comprising a solid state optical detector.

58. The microimaging lens system of claim 57 wherein the solid state optical detector is a CCD array.

59. The microimaging lens system of claim 47 further comprising a structure to block light not originating from the membrane reactor array from reaching the optical detector.

60. The microimaging lens system of claim 59 wherein said structure is an opaque tube comprising a first end which forms a light tight fit to one end of the microimaging lens and comprising a second end which forms a light tight fit the optical detector.

61. The microimaging lens system of claim 59 wherein said structure is an opaque tube comprising a first end which forms a light tight fit to one end of the microimaging lens and comprising a second end which forms a light tight fit to a membrane adaptor array.

62. A sequencing cartridge comprising:
(a) a flow chamber enclosing a membrane reactor array of claim 1 with an inlet port and an outlet port,
(b) an optical window for the optical examination of said membrane reactor array;
(c) an inlet port for delivering sequencing reagents;
(d) a first outlet port for removal of sequencing reagents; and
(e) a second outlet port for removal of effluents.

63. The sequencing cartridge of claim 62 wherein the optical window is circular.

64. The sequencing cartridge of claim 62 wherein the flow chamber is substantially funnel shaped with the wide section of the funnel proximal to the membrane support structure and the narrow section of the funnel at the second outlet port.

65. The sequencing cartridge of claim 62 wherein said support structure comprises a porous solid surface.

66. The sequencing cartridge of claim 62 wherein said inlet is in fluid communication with a pump for controlling a flow of fluids through said inlet.

67. The sequencing cartridge of claim 62 wherein said first outlet port is in fluid communication with a first pump for controlling a flow of fluids through said first outlet port.

68. The sequencing cartridge of claim 62 wherein said second outlet port is in fluid communication with a second pump for controlling a flow of fluids through said second outlet port.

69. The sequencing cartridge of claim 62 wherein said inlet and said first and said second outlet is each in fluid communication with a different pump for controlling a flow of fluids so that a flow of fluids normal to the plane of the membrane reactor array and a flow of fluid tangential to the plane of the membrane reactor array can be controlled simultaneously.

70. A method of producing a membrane reactor array of nucleic acids comprising the steps of:
(a) providing one or more nucleic acid templates to be amplified wherein a plurality of nucleic acid templates are individually attached to separate beads to form a population of nucleic acid template-carrying beads;
(b) suspending said nucleic acid template-carrying beads in an amplification reaction solution containing reagents necessary to perform nucleic acid amplification;
c) forming an emulsion wherein a plurality of said template-carrying beads and PCR reaction solution are individually isolated in the emulsion to form a plurality of microreactors;
(d) amplifying the one or more nucleic acid templates in fluidic isolation from each other to form amplified nucleic acid;
(e) attaching the amplified nucleic acid to said beads; and
(f) loading said beads onto a membrane reactor array of claim 1.

71. A method of producing a membrane reactor array of nucleic acids comprising the steps of:
(a) providing one or more nucleic acid templates to be amplified wherein a plurality of nucleic acid templates are individually attached to separate beads to form a population of nucleic acid template-carrying beads;
(b) loading said beads onto a membrane reactor array of claim 1;
(c) contacting said nucleic acid template-carrying beads to an amplification reaction solution containing reagents necessary to perform nucleic acid amplification; and
(d) amplifying the one or more nucleic acid templates in fluidic isolation from each other to form amplified nucleic acids.


SEQUENCE LISTING (11 sequences)

SEQ ID NO: 1 - DNA, 20 bp, Artificial
  Synthetic Primer
caattaaccctcactaaagg

SEQ ID NO: 2 - DNA, 21 bp, Artificial
  Synthetic Primer
gtaatacgactcactataggg

SEQ ID NO: 3 - DNA, 136 bp, Artificial
  Synthetic Primer
cgatcaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgg

SEQ ID NO: 4 - DNA, 20 bp, Artificial
  Synthetic Probe
caattaaccctcactaaagg

SEQ ID NO: 5 - DNA, 88 bp, Artificial
  Synthetic DNA Sequence
atgccgcaaaaacgcaaaacgcaaacgcaacgcatacctctccgcgtaggcgctcgttggtccagcagaggcggccgcccttgcgcga

SEQ ID NO: 6 - DNA, 20 bp, Artificial
  Synthetic Primer
ccatctgttccctccctgtc

SEQ ID NO: 7 - DNA, 129 bp, Artificial
  Synthetic DNA Template
atgccgcaaaaacgcaaaacgcaaacgcaacgcatacctctccgcgtaggcgctcgttggtccagcagaggcggccgcccttgcgcgagcagaatggcggtagggggtctagctgcgtctcgtccgggg

SEQ ID NO: 8 - DNA, 20 bp, Artificial
  Synthetic Primer
ccatctgttgcgtgcgtgct

SEQ ID NO: 9 - DNA, 20 bp, Artificial
  Synthetic Primer
cgtttcccctgtgtgccttg

SEQ ID NO: 10 - DNA, 84 bp, Artificial
  Synthetic DNA Sequence
cagtcagtcagtcagtcagtcagtcagtcagtcagtcagtcagtcagtcagtcagtcagtcagtcagtcagtcagtcagtcagt

SEQ ID NO: 11 - DNA, 20 bp, Artificial
  Synthetic Amplified DNA
gatcgatcgatcgatcgatc